SEQUENCE LISTING (34 sequences)

SEQ ID NO: 1 - protein, 15 aa, unidentified
KNEQELLELDKWASL

SEQ ID NO: 2 - protein, 123 aa, unidentified
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSS

SEQ ID NO: 3 - protein, 237 aa, synthetic construct
SKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 4 - protein, 50 aa, synthetic construct
DALDDFDLDMLGSDALDDFDLDMLGSDALDDFDLDMLGSDALDDFDLDML

SEQ ID NO: 5 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - RNA, 15 nt, synthetic construct
  source 1..15: mol_type = other RNA
gctagaaagaagaaa

SEQ ID NO: 7 - RNA, 15 nt, synthetic construct
  source 1..15: mol_type = other RNA
gtagtatcaatggag

SEQ ID NO: 8 - RNA, 20 nt, synthetic construct
  source 1..20: mol_type = other RNA
gcatagagctaggggctagt

SEQ ID NO: 9 - RNA, 20 nt, synthetic construct
  source 1..20: mol_type = other RNA
gccggccgcttcaattcact

SEQ ID NO: 10 - RNA, 20 nt, synthetic construct
  source 1..20: mol_type = other RNA
aagtgaagttgacacacgag

SEQ ID NO: 11 - RNA, 20 nt, synthetic construct
  source 1..20: mol_type = other RNA
gggtaaattcccggccatcc

SEQ ID NO: 12 - RNA, 20 nt, synthetic construct
  source 1..20: mol_type = other RNA
gtacgcagtcagcggggaaa

SEQ ID NO: 13 - RNA, 20 nt, synthetic construct
  source 1..20: mol_type = other RNA
tggatgcagtaaaatcgttc

SEQ ID NO: 14 - RNA, 20 nt, synthetic construct
  source 1..20: mol_type = other RNA
tcacgtgcgtagccatacat

SEQ ID NO: 15 - RNA, 20 nt, synthetic construct
  source 1..20: mol_type = other RNA
gccgcgccgcgctatagcac

SEQ ID NO: 16 - RNA, 20 nt, synthetic construct
  source 1..20: mol_type = other RNA
ccgagcagtgcagtacgcgt

SEQ ID NO: 17 - RNA, 20 nt, synthetic construct
  source 1..20: mol_type = other RNA
ttgctgaggattatgacgcg

SEQ ID NO: 18 - RNA, 20 nt, synthetic construct
  source 1..20: mol_type = other RNA
tcgtcgcctattatgggttc

SEQ ID NO: 19 - RNA, 20 nt, synthetic construct
  source 1..20: mol_type = other RNA
agtatgctataccaaggaat

SEQ ID NO: 20 - RNA, 20 nt, synthetic construct
  source 1..20: mol_type = other RNA
atttatagcgtaagcctaca

SEQ ID NO: 21 - RNA, 20 nt, synthetic construct
  source 1..20: mol_type = other RNA
tgtgtagtaagtatatatag

SEQ ID NO: 22 - RNA, 20 nt, synthetic construct
  source 1..20: mol_type = other RNA
ggatttgcattaactcgggt

SEQ ID NO: 23 - RNA, 19 nt, synthetic construct
  source 1..19: mol_type = other RNA
tccagtgtattagtgtggt

SEQ ID NO: 24 - DNA, 341 bp, synthetic construct
  source 1..341: mol_type = other DNA
ggagcaattccgagcaattaagcgtgacagtcccagtgaacccaccgctccattgatactacctaaacgtgatcgcagtccatgcaatcatacgcaaccgtttcttctttctagcaaaggaagttctccatacaccgacgcaccagttggcctctccattgatactactacgccatatacataaacgagctgcacaaatgaaaccatttcttctttctagcgtgtttgaactggacctctagtttctctacaaaaaccactccattgatactacataggttgacctgttgcgaaattgccttgcttagaccctttcttctttctagctgcaatgttgtact

SEQ ID NO: 25 - DNA, 341 bp, synthetic construct
  source 1..341: mol_type = other DNA
ggagttacgactggtacttaatttaatttcgctaacatgatgatccgctccattgatactacctgtcgagtccctcttgtgattggatagttagacaccgtttcttctttctagctatccaggcagtacgtctgtgcccttgaaaaacatcctctccattgatactactactcacctcaatagtaattattcctataacctttccatttcttctttctagcagcaagagcttgcctctctattgatgttacggcgaccactccattgatactacattacttgattagatatactgtctttgacacgttgccctttcttctttctagcataactccgttact

SEQ ID NO: 26 - DNA, 341 bp, synthetic construct
  source 1..341: mol_type = other DNA
ggaggttttatttttatgatccattgatgtccttaaggctgcaaccgctccattgatactacgaactgattatgcctttttctaggattagagtaatccgtttcttctttctagcatgttggagagacagcagcgactgcaaaagatggccctctccattgatactacgatataaacagaaaaatagccgaatatatctattcccatttcttctttctagccgataatcataataggacgcttctaagtttttccaccactccattgatactacattgttttaggagtattattcgagggcattcagaaccctttcttctttctagctcacaaatgatact

SEQ ID NO: 27 - DNA, 125 bp, Arabidopsis sp.
  source 1..125: mol_type = genomic DNA
tactgatttcggtccagggtaaattcgtaatttgcatttaaaccctagttaaacgcattataaaaagacgaaaccctagaagctgagagtgagtgagtgagagaagaatcagagaaaatgcaatg

SEQ ID NO: 28 - DNA, 135 bp, Arabidopsis sp.
  source 1..135: mol_type = genomic DNA
tactggtgttaaaagagttcgattagtaagctttacattttattgatactaccactatatatacgttggaatgtcatatcaaacacagattaacaaattctctcaaatggctctctccgtatttgcctttcaatg

SEQ ID NO: 29 - DNA, 135 bp, Arabidopsis sp.
  source 1..135: mol_type = genomic DNA
tactcaaaggtatcataattatcatcatatgtaatgtagggaacaaatctgtatttactggtatatatatataacacaaaaaaaattaccaaagaaaaaaaaagagcaaagggaacgataccattagtcccaatg

SEQ ID NO: 30 - DNA, 125 bp, Arabidopsis sp.
  source 1..125: mol_type = genomic DNA
tactaattccatgtgcaataataagagtcgatcaccaacttcgctgtgtcactctctctatatataaagactttccctcctttagactttgtgcattatccaaaaaatggagaagaagaagaatg

SEQ ID NO: 31 - DNA, 125 bp, Arabidopsis sp.
  source 1..125: mol_type = genomic DNA
tactgaatttaagatttaagataaatgcaataataaagttccactatttttgagagaaatataaataaaacgcgagattttttattctacgtctaaaaaggctctcacacgattcgttaagaatg

SEQ ID NO: 32 - RNA, 126 nt, Arabidopsis sp.
  source 1..126: mol_type = genomic RNA
tactaccccaattaggattagtgaaaaagtaaaaattaatattaacaaaaatgtaaataaactctttttttgtactttcatcgaaccaaaagacaaaccaggttgcaataaacaaaagcatcaatg

SEQ ID NO: 33 - protein, 5 aa, synthetic construct
GSGSG

SEQ ID NO: 34 - protein, 43 aa, Xanthomonas oryzae
TVMWEQDAAPFAGAADDFPAFNEEELAWLMELLPQSGSVGGTI

(19) United States
(12) Patent Application Publication
(10) Pub. No.: US 2025/0002926 A1
(43) Pub. Date: Jan. 2, 2025

(54) COMPOSITIONS AND METHODS FOR TRANSCRIPTIONAL ACTIVATION

(71) Applicant: REGENTS OF THE UNIVERSITY OF MINNESOTA, Minneapolis, MN (US)

(72) Inventors: Michael Joseph Smanski, Falcon Heights, MN (US); Juan Armando Casas Mollano, Minneapolis, MN (US)

(21) Appl. No.: 18/708,411

(22) PCT Filed: Nov. 10, 2022
(86) PCT No.: PCT/US2022/049494

§ 371 Date: May 8, 2024

Related U.S. Application Data
(60) Provisional application No. 63/278,790, filed on Nov. 12, 2021.

(51) Int. Cl. C12N 15/82; C12N 9/22; C12N 15/11

(52) U.S. Cl. CPC C12N 15/8213; C12N 9/22; C12N 15/11; C12N 15/8205; C12N 2310/20

(57) ABSTRACT

A transcriptional activator system including a dCas protein, a nanobody, a binding polypeptide, and a sgRNA. The binding polypeptide is fused to the dCas protein. The binding polypeptide includes a binding sequence that is designed to bind to the nanobody. In one or more embodiments, the nanobody is llama GP41 and the amino acid binding sequence is GP41. In one or more embodiments, a synthetic promoter may be used to influence transcription of a target gene. The synthetic promoter includes a core promoter and a trans-activation region. The trans-activation region includes at one sgRNA binding site.

### CROSS-REFERENCE TO RELATED APPLICATION

This application claims the benefit of U.S. Provisional Patent Application No. 63/278,790, filed Nov. 12, 2021, which is incorporated herein by reference in its entirety.

### GOVERNMENT FUNDING

This invention was made with government support under 151437MOD7 awarded by the Defense Advanced Research Projects Agency and 2018-33522-28747 awarded by the National Institute of Food and Agriculture, USDA. The government has certain rights in the invention.

### SEQUENCE LISTING

This application contains a Sequence Listing electronically submitted to the United States Patent and Trademark Office via Patent Center as an XML file entitled “0110_000688WO01” having a size of 33.3 kilobytes and created on Nov. 9, 2022. Due to the electronic filing of the Sequence Listing, the electronically submitted Sequence Listing serves as both the paper copy required by 37 CFR § 1.821 (c) and the CRF required by § 1.821 (e). The information contained in the Sequence Listing is incorporated by reference herein.

### SUMMARY

This disclosure describes, in one aspect, a transcriptional activator system. Generally, the transcriptional activator system includes a dCas protein, a nanobody, a binding polypeptide, and a sgRNA.

In another aspect, this disclosure describes a transcriptional activator system. Generally, the transcriptional activator system includes a first nucleotide sequence that encodes a dCas protein, a second nucleotide sequence that encodes a binding polypeptide, a third nucleotide sequence that encodes a nanobody, a fourth nucleotide sequence that encodes an activator domain, and a fifth nucleotide sequence that encodes a sgRNA.

In another aspect, this disclosure describes a method. The method includes providing a cell with a with a first nucleotide sequence that encodes a dCas protein, a second nucleotide sequence that encodes a binding polypeptide, a third nucleotide sequence that encodes a nanobody, a fourth nucleotide sequence that encodes an activator domain, and a fifth nucleotide sequence that encodes a sgRNA sequence. The method further includes allowing the cell to express the dCas protein, express the binding polypeptide, express the nanobody, transcribe the sgRNA, integrate the sgRNA with the dCas protein, pair the sgRNA with a target sequence, and initiate transcription of a target gene.

In all the above aspects, the nanobody includes and activator domain.

In all the above aspects, the binding polypeptide includes an amino acid binding sequence that is designed to bind to the nanobody.

In all the above aspects, the binding polypeptide is fused to the dCas protein.

In some embodiments, the nanobody is llama GP41 (SEQ ID NO: 2) and the amino acid binding sequence is GP41 (SEQ ID NO: 1).

In some embodiments, nanobody further includes a solubilizing domain. In some embodiments, the solubilizing domain includes GB1, sfGFP, or both.

In some embodiments, the activator domain is VP64 (SEQ ID NO: 4), TAL (SEQ ID NO: 34), or a combination thereof.

In some embodiments, the dCas protein is dCas9 (SEQ ID NO: 5).

In some embodiments, the binding polypeptide includes at least five copies of the amino acid binding sequence.

In yet another aspect, the present disclosure describes a synthetic promoter for influencing the transcription of a target gene. Generally, the synthetic promoter includes a core promoter and a trans-activation region. The core promoter includes a first region and a second region. The first region is 15-20 nucleotides downstream from the transcription initiation site of the target gene. The second region is 80-85 nucleotides upstream from the transcription initiation site of the target gene. The trans-activation region is upstream from the transcription initiation site of the target gene and includes a least one sgRNA binding site.

In some embodiments, the core promoter includes an Arabidopsis promoter. In some embodiments, Arabidopsis core promoter includes SEQ ID NO: 27, SEQ ID NO: 28, SEQ ID NO: 29, SEQ ID NO: 30, SEQ ID NO: 31, or SEQ ID NO: 32.

In some embodiments, the trans-activation region comprises one or more additional sgRNA binding sites.

In some embodiments, the trans-activation region is SEQ ID NO. 24, SEQ ID NO: 25, or SEQ ID NO: 26.

In some embodiments, any synthetic promoter of the previous aspects and/or embodiments and any transcriptional activator systems of the previous aspects and/or embodiments are used in a method. The method includes integrating the synthetic promoter of any one of the previous aspects and/or embodiments into the genome of a cell and providing the cell any one of the transcriptional activator systems of the previous aspects and/or embodiments. The method includes allowing the cell to express the dCas protein, express the peptide, express the nanobody, transcribe the sgRNA, integrate the sgRNA with the dCas protein, pair the sgRNA with the sgRNA binding site, and initiate transcription of the target gene. In some embodiments, the cell is a plant cell. In some embodiments, the synthetic promoter includes a core promoter selected from the group consisting of SEQ ID NO: 24, ID NO: 25 and SEQ ID NO: 26; and a trans-activating region selected from the group consisting of SEQ ID NO: 27, SEQ ID NO: 28, SEQ ID NO: 29, SEQ ID NO: 30, SEQ ID NO: 31 and SEQ ID NO: 32. In some embodiments, the method further includes integrating one or more synthetic promoters to influence the transcription of one or more additional target genes in the genome of the cell. In some embodiments, the method further includes providing the cell with one or more sgRNA designed to pair with the one or more sgRNA binding sites on the one or more transcription activation region.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Expression of scFv and NbGP41 fusions in Setaria protoplasts. Top panels: GFP (left) and bright field (right) microscopy of protoplasts transformed with scFv-sfGFP-VP64-GB1. Lower panels: GFP (left) and bright field (right) microscopy of protoplasts transformed with NbGP41-sfGFP-VP64-GB1. GFP: Image taken using fluorescent microscopy. BF: image taken using bright field microscopy.

FIG. 2. Schematic representation of the MoonTag activator. Left: Diagrams of the expression constructs encoding the protein components of MoonTag. The DNA binding component contains dCas9 (SEQ ID NO:5) fused to a binding polypeptide that includes ten copies of the GP41 amino acid binding sequence (SEQ ID NO:1) (dCas9-10XGP41); an activation module including the nanobody GP41 (SEQ ID NO:2) fused to sfGFP (super folder GFP) (SEQ ID NO:3), the VP64 activation domain (SEQ ID NO:4), and the GB1 solubility tag (NbGP41:sfGFP:VP64:GB1); and finally the sgRNA expression cassette driven by a U6 promoter. Right: When expressed in plant cells dCas9-10XGP41 binds to DNA guided by the sgRNA. The GP41 amino acid binding sequence (SEQ ID NO: 1) copies in dCas9-10XGP41 are bound by the GP41 nanobody (SEQ ID NO:2) of NbGP41-sfGFP-VP64-GB1 recruiting up to ten copies of the VP64 (SEQ ID NO:4) activation domains to the ribonucleoprotein complex.

FIG. 3. Schematic representation of the luciferase reporter used to test the activity of MoonTag and SunTag in Setaria protoplasts. Top: the luciferase gene is driven by a promoter containing the minimal promoter of CaMV 35S. Six copies of a sgRNA binding site (sgRNA BS) were cloned upstream of the minimal promoter. Bottom: By itself, the promoter is inactive. Transcription is stimulated when the transcription activator binds to the sgRNA binding site, thereby initiating luciferase production, the presence of which is detected by luminescence in the presence of D-luciferin.

FIG. 4. Activation of gene expression by CRISPR-Cas activators in Setaria protoplasts. (A) Activation of a luciferase reporter by SunTag and MoonTag in Setaria protoplasts. Expression of SunTag without the scFv component is used as control. 10× and 24× indicates the copy number of the GCN4 or the GP41 amino acid binding sequences on the binding polypeptide that is fused to dCas9. ST indicates the SunTag component. MT indicates the Moon Tag component. Ab-sfGFP-VP64-GB1 referrers to scFv in SunTag (ST-GFP) or NbGP41 in MoonTag (MT1) fused to sfGFP-VP64-GB1. Ab-VP64-GB1 referrers to scFv in SunTag (ST-v1) or NbGP41 in MoonTag (MT2) fused to VP64-GB1. Nb-VP64-GB1 refers to NbGP41 fused to VP64 (MT3). (B) Activation of the indicated endogenous genes by MoonTag in Setaria protoplasts. NOG: Expression of MoonTag without the targeting sgRNA is used as a control. MT2-10×: is the MoonTag expressing dCas9-10XGP41 with NbGP41-VP64-GB1 and three sgRNAs for the target gene. MT2-24×: is the MoonTag expressing dCas9-24XGP41 with NbGP41-VP64-GB1 and three sgRNAs for the target gene.

FIG. 5. Expression of MoonTag in Setaria transgenic plants. (A) T-DNA of the binary vector constructed to express the MoonTag activator in Setaria. The genes encoding the different components of MoonTag are indicated. dCas9-24XGP41 and NbGP41-sfGFP-VP64-GB1 are driven by the CmYLCV promoter. The CLV3 sgRNA, indicated as sgRNA3, is driven by the rice U6 promoter. Expression of the selectable marker HPTII is driven by the switchgrass UBI2 promoter. A Luciferase reporter driven by the 35S promoter was also included in the binary vector. Right border (RB) and left border (LB) of the T-DNA are represented by grey rhombuses. (B) Expression of MoonTag components in four putative transgenic plants. The expression of dCas9-24XGP41 and NbGP41-sfGFP-VP64 was calculated relative to the endogenous gene GRAS. (C) GFP signal from NbGP41-sfGFP-VP64-GB1 in protoplasts prepared from leaves a transgenic plant expressing the MoonTag construct. GFP: Fluorescent microscopy. BF: bright field microscopy.

FIG. 6. Expression of CLV3 and MoonTag components in homozygous T2 Setaria transgenic plants. The genes analyzed are indicated on top of each graphic. For each transgenic line, sgRNA3-1, and sgRNA3-3, the leaves of four homozygous individuals were analyzed. CLV3 expression is expressed as an average of four individuals for each line and normalized to its expression in the wild type Me34. For the remaining lines the expression of each individual is shown in the graph while the line represents the average. Expression of the GRAS gene was used a reference.

FIG. 7. Luciferase activity in tomato hairy roots transformed with the indicated activators. (A) T-DNA of the binary vector constructed to express MoonTag activating a luciferase reporter in tomato. The genes encoding the different components of MoonTag are indicated. dCas9-24XGP41 and NbGP41-sfGFP-VP64-GB1 are driven by the AtUBI10 promoter. The gRNAs, indicated as gRNA1 and gRNA2, are driven by the Arabidopsis U6 promoter. Expression of the selectable marker NPTII is driven by the 2× 35S promoter. The Luciferase reporter is driven by synthetic promoter with binding site for gRNA1 and gRNA2. Right (RB) and left borders (LB) of the T-DNA are represented by rhombuses. (B) K599, tomato hairy root without any activator or luciferase transgene. MT13X-NOG, Moontag components without a sgRNA; ST1-10X, SunTag with 10× GCN4 copies; MT1-13X MoonTag with 13 copies of GP41; MT1-24X, Moontag with 24× copies of GP41. Luminescence signal captured one month after transformation with a CCD camera superimposed on a bright field image of tomato hairy roots.

FIG. 8. Luciferase expression in tomato hairy roots transformed with the indicated activators. (A) Expression of luciferase measured by RT-qPCR in the different hairy roots lines (B) Expression of luciferase in the two independent lines transformed with the indicated constructs after 10 months of subculture. K599, tomato hairy root without any activator or luciferase transgene. MT1-13X-NOG, Moontag components without a gRNA; ST1-10X, SunTag with 10× GCN4 copies; MT1-13X MoonTag with 13 copies of GP41; MT1-24X, Moontag with 24× copies of GP41. Each point in the graph represents an independent transformation event. Gene expression was normalized to that of Actin2.

FIG. 9. Expression of MoonTag and SunTag in tomato hairy roots. (A) Expression analysis of the MoonTag and SunTag components. dCas9-peptide refers to the expression of the DNA binding component fused to the GCN4 peptide in SunTag or the GP41 peptide in MoonTag. Ab-VP64 referrers to the antibody fusion to the activation domain, scFv in SunTag (ST-GFP) or NbGP41 in MoonTag. MT1-13X-NOG, Moontag components without a sgRNA; STSJ, SunTag with 10× GCN4 copies; MT1-13X MoonTag with 13 copies of GP41; MT1-24X, Moontag with 24× copies of GP41. Gene expression was normalized to that of Actin2. (B) Expression of NbGP41 and scFv antibody fusions with sfGFP-VP64-GB1 in tomato hairy roots. Pictures are representative of the GFP signal observed in two hairy roots lines for MoonTag (MT1-13X) and SunTag (STSJ-10X) expressing similar mRNA levels of the antibody-sfGFP-VP64-GB1 fusions.

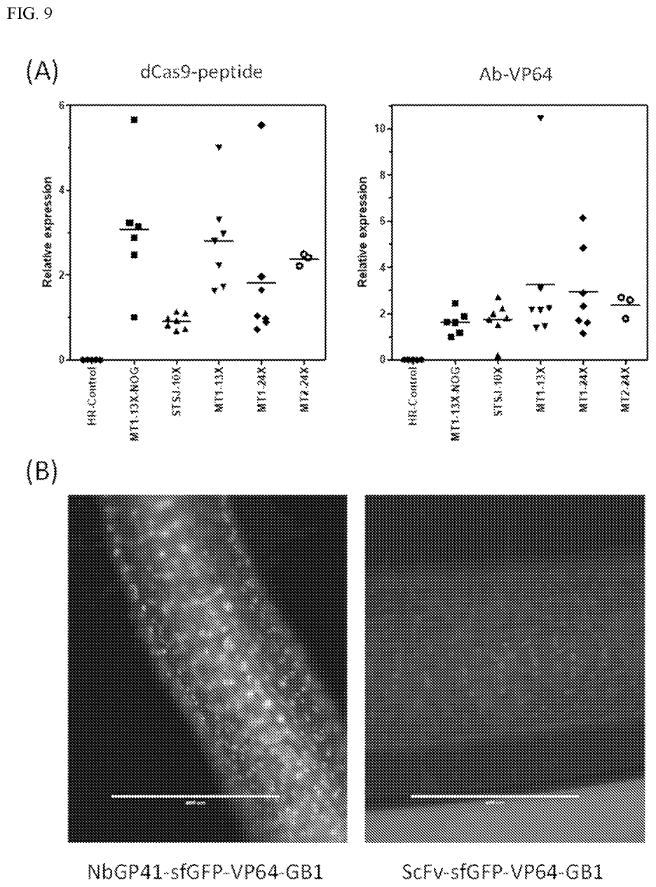

FIG. 10. MoonTag is capable of activating endogenous genes in transgenic Arabidopsis plants. (A) Diagram of the FT gene showing the position of the gRNAs in relation to the TSS. (B) Left: RT-qPCR expression of the FT gene in the indicated transgenic lines. Right: Total rosette leaf number until flowering shown by each transgenic line. Expression of the FT gene was quantified in three seedlings from plants homozygous for the transgenes. (C) Flowering phenotype of the indicated transgenic lines.

FIG. 11. MoonTag is capable of activating endogenous genes in transgenic Arabidopsis plants. (A) Diagram of the CLV3 gene indicating the position of the gRNAs designed to activate this gene. (B) RT-qPCR expression of the CLV3 in the indicated lines. Expression of the CLV3 gene was quantified in three seedlings from plants homozygous for the transgenes. (C) Phenotype resulting from the overexpression of CLV3.

FIG. 12. MoonTag activation is reduced at lower temperature in Arabidopsis and tomato. (A) Activation of CLV3 by MoonTag in Arabidopsis seedlings incubated at different temperatures. (B) Luciferase expression in hairy roots incubated at the indicated temperatures.

FIG. 13. Expression of the components that make up MoonTag (dCas9, top; co-activator, middle; gRNA, bottom) at different temperatures. Expression was normalized to that of TUB2 for Arabidopsis (left) and to Actin2 for tomato hairy roots (right).

FIG. 14. Activation of endogenous genes by MoonTag in Arabidopsis. (A) Expression of FT in the wild type col-0 and 16 transgenic lines expressing MoonTag and two sgRNAs targeting the FT promoter. (B) Expression of CLV3 in the wild type col-0 and 13 transgenic lines expressing MoonTag and two sgRNAs targeting the CLV3 promoter. Expression was normalized to that of TUB2.

FIG. 15. Activation of a luciferase reporter in Setaria protoplasts transiently expressing MoonTag with various activation domains AD1-AD6. AD1 is TAL. AD4 is VP64. AD2, AD3, AD5, and AD6 are modified version of a known activator domains. Luciferase activity was normalized to that of MoonTag with a VP64 activation domain.

FIG. 16. Characterization of synthetic promoters in Setaria protoplasts. (A) Schematic representation of the synthetic promoter driving the luciferase reporter. The minimal promoter or core promoter is represented by a grey bar. sgRNA1 and sgRNA2 binding sites are represented by green and red triangles respectively. (B) Luciferase activity of the synthetic promoters assembled with the indicated core promoters when transformed together with MoonTag with and without the presence of the sgRNA expression cassettes. (C) Luciferase activity of the indicated synthetic promoters in the presence of either sgRNA1 (SEQ ID NO:6), sgRNA2 (SEQ ID NO:7), both or the absence of them.

FIG. 17. Activation of the betalain biosynthesis pathway by SunTag and MoonTag. (A) Representative Nicotiana benthamiana leaf agroinfiltrated with Agrobacterium strains carrying the pBet-1 and pST-v1 binary vectors. (B) Representative Nicotiana benthamiana leaf agroinfiltrated with Agrobacterium strains carrying the pBet-1 and pMT2 binary vectors. (C) Expression of betalain biosynthesis genes, CYP76, DODA and GT, in the presence and absence of the SunTag activator. Gene expression was normalized to that of Actin2 and then to the expression of each target gene in the sample infiltrated with the pBet-1 plasmid only.

FIG. 18. Vector map of pMod_A-CmYLCV-dCas9-24XGP41-HSP-ter. This vector contains the coding sequence of dCas9-24XGP41 driven by the CmYLCV promoter. The 3′ end polyadenylation signal is provided by the HSP terminator. The open reading frame of dCas9-24XGP41 is shown by a green line.

FIG. 19. Vector map of pMod_D-CmYLCV-NbGP41-sfGFP-VP64-GB1-RBCS-ter. This vector contains the coding sequence of NbGP41-sfGFP-VP64-GB1 driven by the CmYLCV promoter. The 3′ end polyadenylation signal is provided by the RBCS terminator. The open reading frame of NbGP41-sfGFP-VP64-GB1 is shown by a green line.

FIG. 20. Vector map of pMod_D-cmYLCV-NbGP41-VP64-GB1-RBCSter. This vector contains the coding sequence of NbGP41-VP64-GB1 driven by the CmYLCV promoter. The 3′ end polyadenylation signal is provided by the RBCS terminator. The open reading frame of NbGP41-VP64-GB1 and of the amp resistance genes are indicated by a green line.

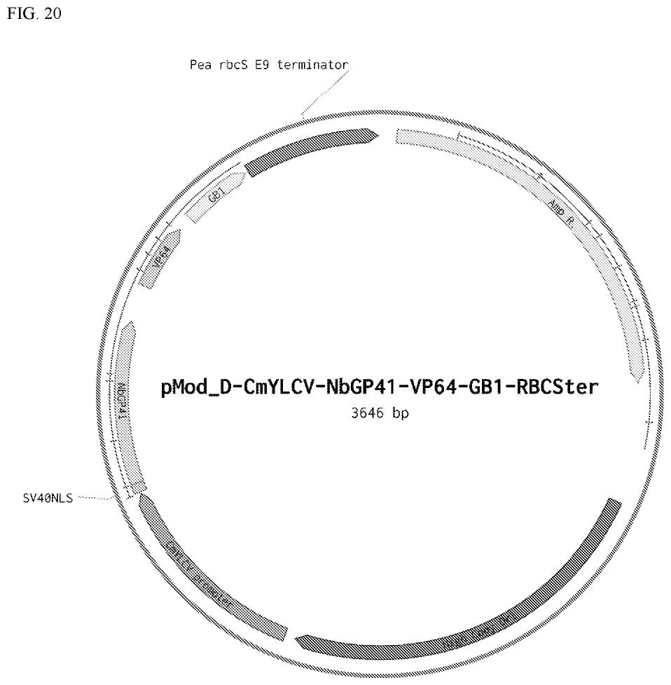

FIG. 21. Vector map of pMod_B-OsU6-sgRNA1. This vector contains the spacer corresponding to the sgRNA1 follow by sgRNA scaffold driven by rice U6 promoter.

FIG. 22. Vector map of pMod_C′-MTAP-Luciferase-RBCS-ter. This vector contains the coding sequence of the firefly luciferase driven by the MTAP promoter that contains six binding sites for the sgRNA1. The 3′ end polyadenylation signal is provided by the RBCS terminator.

FIG. 23. Vector map of pMod_B-AtU6-sgRNA1-AtU6-sgRNA2. This vector contains two sgRNAs expression cassettes separated by an unrelated sequence (SPCR). The first sgRNA cassette contains the spacer corresponding to the sgRNA1 follow by sgRNA scaffold driven by the Arabidopsis U6 promoter. The second sgRNA cassette contains the spacer corresponding to the sgRNA2 follow by sgRNA scaffold driven by the Arabidopsis U6 promoter.

FIG. 24. Vector map of pMod_A-TA1-P0670-CYP76-OCS-ter. This vector contains the coding sequence of the CYP76 gene driven by a synthetic promoter made by combining TA1 (SEQ ID NO:24; containing three binding sites for sgRNA1 and three binding sites for sgRNA2) and the core promoter P0670 (SEQ ID NO:27). The 3′ end polyadenylation signal is provided by the OCS terminator.

FIG. 25. Vector map of pMod_C′-TA2-P1500-DODA-MAS-ter. This vector contains the coding sequence of the DODA gene driven by a synthetic promoter made by combining TA2 (SEQ ID NO:25; containing three binding sites for sgRNA1 and three binding sites for sgRNA2) and the core promoter P1500 (SEQ ID NO:28). The 3′ end polyadenylation signal is provided by the MAS terminator.

FIG. 26. Vector map of pMod_D-TA3-P8470-GT-35S-ter. This vector contains the coding sequence of the DODA gene driven by a synthetic promoter made by combining TA3 (SEQ ID NO:26; containing three binding sites for sgRNA1 and three binding sites for sgRNA2) and the core promoter P8470 (SEQ ID NO 31). The 3′ end polyadenylation signal is provided by the 35S terminator.

### DETAILED DESCRIPTION OF ILLUSTRATIVE EMBODIMENTS

This disclosure describes a transcription activator system that is effective for regulating gene expression in plants. The transcriptional activator system is a modification of the CRISPR-dCas system.

CRISPR-Cas-based transcriptional activators have been developed to induce gene expression in eukaryotic organisms. CRISPR-Cas-based activators include two main components. The first component is the DNA binding domain, which includes a catalytically inactive or nuclease “dead” Cas (dCas) protein. The second component is an activation domain (AD) that can stimulate transcription when associated with a core promoter region. CRISPR-Cas-based systems can achieve high levels of transcriptional activation. Additionally, CRISPR-Cas systems are programable by pairing the guide RNA (sgRNA) and the DNA target strand.

The first generation of CRISPR-Cas activators was created by the direct fusion of the VP64 activation domain to the C-terminus of the dCas9 protein. The resulting activator induced the expression of reporters and endogenous genes at only moderate levels. A more efficient, second generation of CRIPR-Cas activators was created by constructing systems that recruit multiple activation domains, either identical or different, to the promoter regions.

One of these second generation CRISPR-Cas9 activators is SunTag, a system that induces transcription by recruiting multiple copies of an activation domain using the antigen-antibody interaction between a single-chain fragment variable (scFv, fused to the VP64 activation domain) and a 19-amino-acid peptide (fused to the dCas9 protein). SunTag was designed to contain ten copies of the GCN4 peptide fused to dCas9, potentially allowing recruitment of up to ten copies of the VP64 domain to the target promoter. When tested in Arabidopsis, SunTag showed strong transcriptional activation of endogenous genes with the occurrence of the phenotypes expected from the ectopic expression of the target genes.

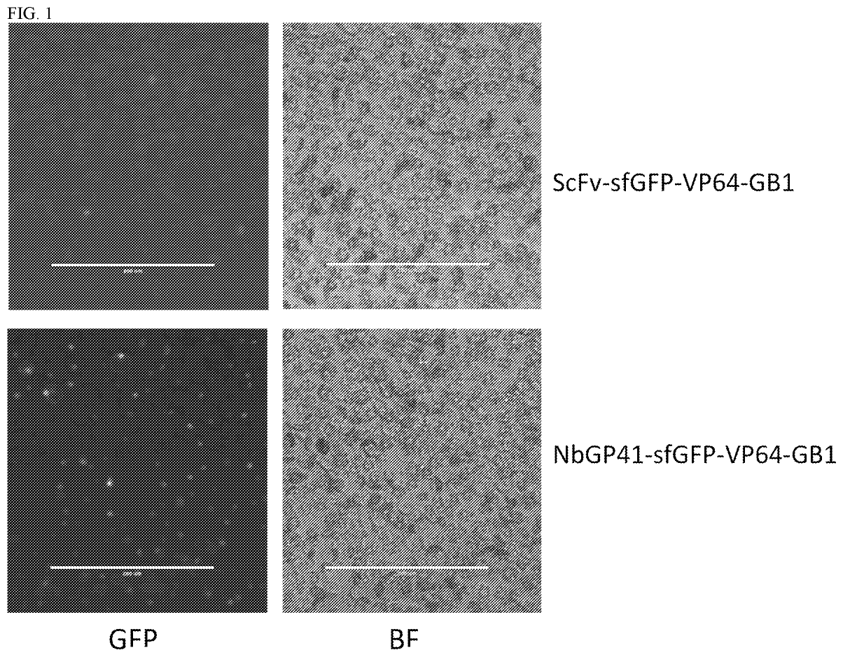

Although SunTag is an efficient activator in Arabidopsis, SunTag is difficult to stably express in transgenic plants such as in the monocot plant Setaria. In the SunTag system, the scFv antibody was already optimized for intracellular expression. However, the scFv antibody showed signs of aggregation when expressed in mammalian cells. Therefore, the scFv antibody needed to be fused together with sfGFP and GB1 tags to increase its solubility. However, scFv expression with the solubility tags may still be an issue in Setaria. The SunTag activating component, scFv-sfGFP-VP64-GB1, seems to be poorly expressed when transiently expressed in protoplasts (FIG. 1, top panel).

### Transcriptional Activator System

In one aspect, this disclosure describes a CRISPR-Cas transcriptional activating system, namely an activator system that exploits MoonTag-type nanobody-peptide interactions. In comparison to the SunTag activator system, the components of the MoonTag activator system are better tolerated when stably expressed in transgenic plants. MoonTag replaces the antibody-peptide interaction (e.g., scFv-GCN4) of SunTag with a nanobody-peptide (e.g., NbGP41-GP41) interaction to recruit the VP64 activation domain (SEQ ID NO:4). In one or more embodiments, the GP41 nanobody (SEQ ID NO:2) is a llama nanobody. Since nanobodies are smaller and more soluble than scFvs, the NbGP41-sfGFP-GB1 fusion is readily expressed in Setaria protoplasts (FIG. 1, bottom panel).

Generally, the MoonTag activating system includes a DNA binding component and an activation module.

The DNA binding component includes a ribonucleoprotein complex and a binding polypeptide. The ribonucleoprotein complex includes a dead Cas (dCas) protein and guide RNA (sgRNA) complexed with the dCas. The DNA binding component also includes a binding polypeptide (e.g., GP41, SEQ ID NO:1). The binding polypeptide is fused to the dCas protein.

The ribonucleoprotein includes a dead Cas (dCas) protein. As used herein, dead Cas refers to a nuclease-inactive Cas protein. Any dCas protein may be used. Examples of dCas proteins include, but are not limited to, dCas3, dCas8, dCas9, dCas10, dCas12, and dCas13. In one or more embodiments, the dCas protein is dCas9 (SEQ ID NO:5). In one or more embodiments, the dCas protein may a part of a larger protein complex.

The ribonucleoprotein includes sgRNA. The sgRNA is complexed with the dCas protein. The sgRNA is generally designed to recognize and bind to a target DNA sequence. The sgRNA generally binds to the target DNA sequence through nucleotide base pairing interactions such Watson and Crick hydrogen bonding. The target DNA sequence may be a promoter region, a trans-activation region, or an enhancer region.

The DNA binding component includes a binding polypeptide. The binding polypeptide includes an amino acid binding sequence that is generally designed to provide a binding interface with a nanobody. The binding polypeptide is fused to the dCas protein. In one or more embodiments, the binding polypeptide includes two or more copies of the amino acid binding sequence. In one or more embodiments, the binding polypeptide includes two or more of the same amino acid binding sequence. In one or more embodiments, the binding polypeptide includes two or more different amino acid binding sequences. In one or more embodiments, the binding polypeptide includes at least two of the same amino acid binding sequence and at least one different amino acid binding sequence. In one or more embodiments amino acid binding sequence is GP41 (SEQ ID NO:1) or a structurally similar peptide.

As used herein, a polypeptide is “structurally similar” to a reference polypeptide if the amino acid sequence of the polypeptide possesses a specified amount of identity compared to the reference polypeptide. Structural similarity of two polypeptides can be determined by aligning the residues of the two polypeptides (for example, a candidate polypeptide and the polypeptide of, for example, SEQ ID NO: 1, to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide (e.g., SEQ ID NO:1). A candidate polypeptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, biologically active analogs of a polypeptide containing deletions or additions of one or more contiguous or noncontiguous amino acids that do not eliminate a functional activity of the polypeptide are also contemplated.

A binding polypeptide as described herein can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to the reference amino acid sequence.

A binding polypeptide as described herein can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

In one or more embodiments, the binding polypeptide has at least two, at least five, at least ten, at least 15, at least 20, at least 25, or at least 30 copies of the amino acid binding sequence. In one or more embodiments, the binding polypeptide has no greater than 50, no greater than 30, no greater than 25, no greater than 20, no greater than 15, no greater than 10, or no greater than five copies of the amino acid binding sequence. In one or more embodiments, the binding polypeptide has five to 50, five to 30, five to 25, five to 20, five to 15, or five to 10 copies of the amino acid binding sequence. In one or more embodiments, the binding polypeptide has ten to 50, ten to 30, ten to 25, ten to 20, or ten to 15 copies of the amino acid binding sequence. In one or more embodiments, the binding polypeptide has 15 to 50, 15 to 30, 15 to 25, or 15 to 20 copies of the amino acid binding sequence. In one or more embodiments, the binding polypeptide has 20 to 50, 20 to 30, or 20 to 25 copies of the amino acid binding sequence. In one or more embodiments, the binding polypeptide has 25 to 50 or 25 to 30 copies of the amino acid binding sequence. In one or more embodiments, the binding polypeptide has 30 to 50 copies of the amino acid binding sequence. In one or more embodiments, the binding polypeptide has 10 copies of the amino acid binding sequence. In one or more embodiments, the binding polypeptide has 23 copies of the amino acid binding sequence.

The binding polypeptide may include a spacer amino acid sequence between the amino acids of otherwise adjacent binding sequences. Spacer amino acid sequences can have from 5 to 25 amino acids. In one or more embodiments the spacer amino acid sequence is GSGSG (SEQ ID NO: 33) (also known as a GS linker) In one or more embodiments, spacer amino acid sequences in the binding polypeptide are all the same. In one or more embodiments, the spacer amino acid sequences in the binding polypeptide are all different. In one or more embodiments, at least two spacer amino acid sequences are the same and at least one spacer amino acid sequence is different.

The binding polypeptide may include a N-terminal spacer sequence at the location where the binding polypeptide is fused to the Cas9 protein. The N-terminal spacer sequence may be the same as the spacer amino acid sequence. The N-terminal spacer sequence may be different than the spacer amino acid sequence. In one or more embodiments, the N-terminal spacer sequence is longer than the amino acid spacer sequence. In one or more embodiments, the N-terminal spacer sequence is shorter than the amino acid spacer sequence. In one or more embodiments the N-terminal spacer amino acid sequence is GSGSG (SEQ ID NO:33). In one or more embodiments, the N-terminal spacer amino acid sequence is a nuclear localization signal.

The MoonTag activation system includes an activation module. The activation module includes a nanobody. The nanobody includes a recognition domain that is capable of binding to the amino acid binding sequence. The nanobody includes an activator domain. The activator domain is designed to promote transcription of a target gene. The activator domain may be any DNA sequence, RNA sequence, or protein that promotes transcription of a target gene. Examples of activator domains include VP64 (SEQ ID NO:4), p65, Rta, GCN4, TAL (avrXa10 gene of Xanthomonas oryzae pv. Oryzae; SEQ ID NO:34), ERF2m, EDLL, and Arabidopsis cold binding factor 1 (CBF1). In one or more embodiments the activator domain is GCN4. In one or more embodiments, the activator domain is TAL (SEQ ID NO:34). In one or more embodiments, the activator domain is VP64 (SEQ ID NO:4)

In one or more embodiments, one or more solubility tags are fused to the nanobody. Any solubility tag that does not destroy the ability of the nanobody to bind to the amino acid binding sequence and that does not destroy the ability of the activator domain to activate transcription is contemplated. Examples of solubility tags include super folding green fluorescent protein (sfGFP), glutathione-S-transferase (GST), thioredoxin (Trx), IgG-binding domain from protein A (Z-tag), disulphide isomerase I (DsbA), small ubiquitin-related modifier (SUMO), immunoglobulin-binding domain of protein G (GB1), inactive bacterial haloakane dehalogenase (HaloTag7), and FLAG-tag. In one or more embodiments, the solubility tag is GB1. In one or more embodiments, the solubility tag is sfGFP (SEQ ID NO:3).

In one or more embodiments, the primary function of an sfGFP (SEQ ID NO:3) tag may be something other than increasing the solubility of the activation domain. For example, in one or more embodiments, the primary function of the sfGFP (SEQ ID NO:3) tag is to provide a visible signal. However, a secondary function of an sfGFP (SEQ ID NO:3) tag may be to increase the solubility of the activation domain. Other proteins may be used as tags to provide visible signals including RFP and mCherry.

In one or more embodiments, at least one, at least two, at least three, at least four, or at least five solubility tags are fused to the nanobody. In one or more embodiments, no greater than six, no greater than five, no greater than four, no greater than three, or no greater than two solubility tags are fused to the nanobody. In one or more embodiments, two to six, two to five, two to four, or two to three solubility tags are fused to the nanobody. In one or more embodiments, three to six, three to five, or three to four solubility tags are fused to the nanobody. In one or more embodiments, four to six or four to five solubility tags are fused to the nanobody. In one or more embodiments, five to six solubility tags are fused to the nanobody. In one or more embodiments when more than one solubility tag is fused to the nanobody, all the solubility tags are the same. In one or more embodiments when more than one solubility tag is fused to the nanobody, all the solubility tags are the different. In one or more embodiments when more than one solubility tag is fused to the nanobody, at least two of the solubility tags are the same and at least one solubility tag is different. In one or more embodiments, two solubility tags are fused to the nanobody. In one or more embodiments, the two solubility tags are GB1 and sfGFP (SEQ ID NO: 3).

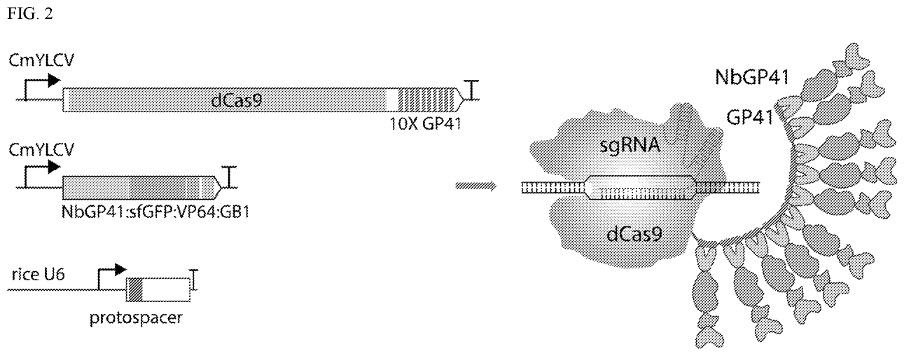

FIG. 2 illustrates an exemplary MoonTag activating system includes a DNA binding component and an activation module. In this illustrative embodiment, the DNA binding component includes dCas9 (SEQ ID NO:5). The dCas9 (SEQ ID NO:5) is fused to the binding polypeptide that includes ten copies of the binding amino acid sequence GP41 (dCas9-10XGP41). The DNA binding component of the MoonTag activation system also includes a guide RNA (sgRNA). The activation module includes a GP41 nanobody (SEQ ID NO:2). The GP41 nanobody (SEQ ID NO:2) is fused to sfGFP (SEQ ID NO:3), the solubility tag GB1, and the VP64 activation domain (SEQ ID NO:4) (NbGP41-sfGFP-VP64-GB1). The MoonTag activation system includes a guide RNA (sgRNA).

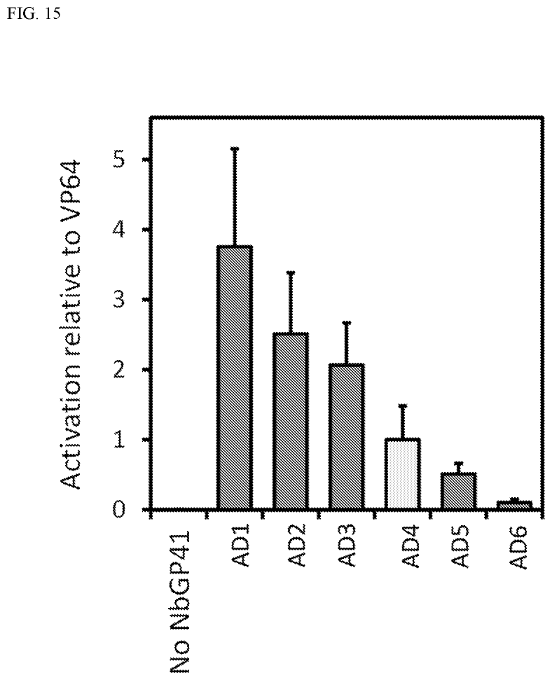

When expressed in plant cells dCas9-10XGP41 binds to its target regions guided by the sgRNA. The binding polypeptide that includes the GP41 amino acid binding sequences (SEQ ID NO: 1) in dCas9-10XGP41 are bound by the GP41 nanobody (SEQ ID NO:2) of NbGP41-sfGFP-VP64-GB1 recruiting up to ten copies of the VP64 activation domains (SEQ ID NO:4) to the ribonucleoprotein complex (FIG. 2). To begin testing MoonTag, several variations of the components were developed and tested to find the best combination giving optimal activation. Variations included dCas9 fused to different number of repeats of the GP41 amino acid binding sequence (10, 13, and 24 copies; SEQ ID NO:1). Additionally, versions of the activation module without sfGFP (FIG. 15) and without both sfGFP (SEQ ID NO:3) and GB1 (NbGP41-VP64) were tested.

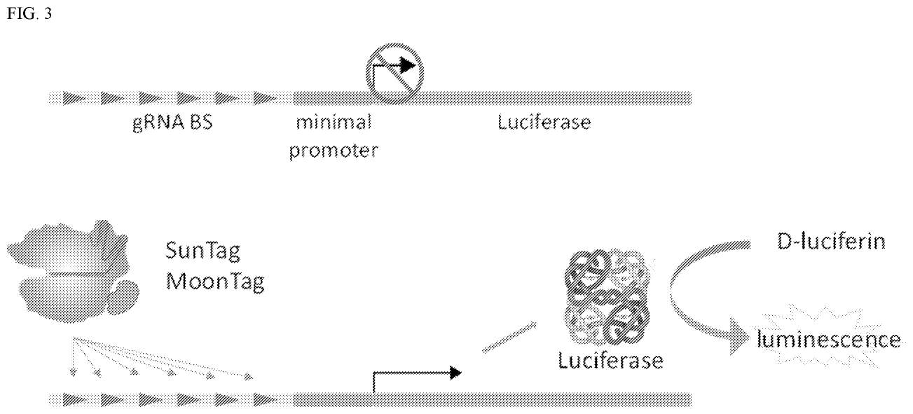

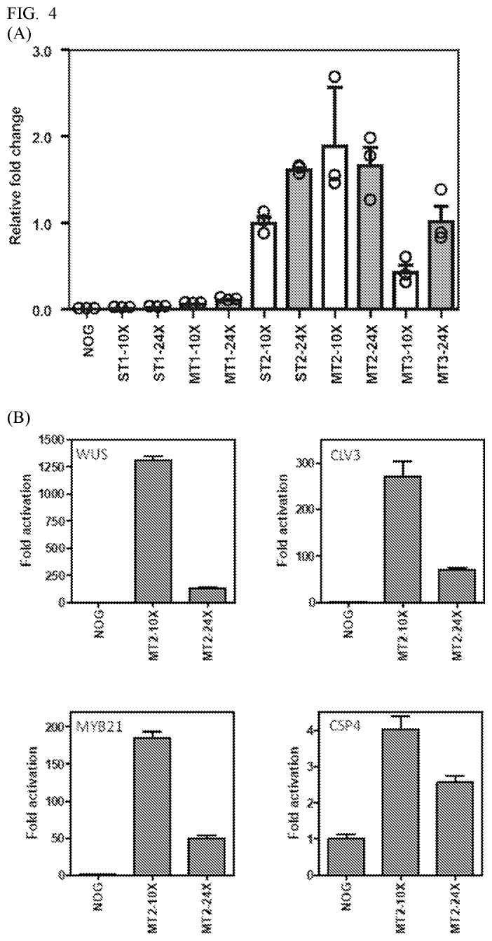

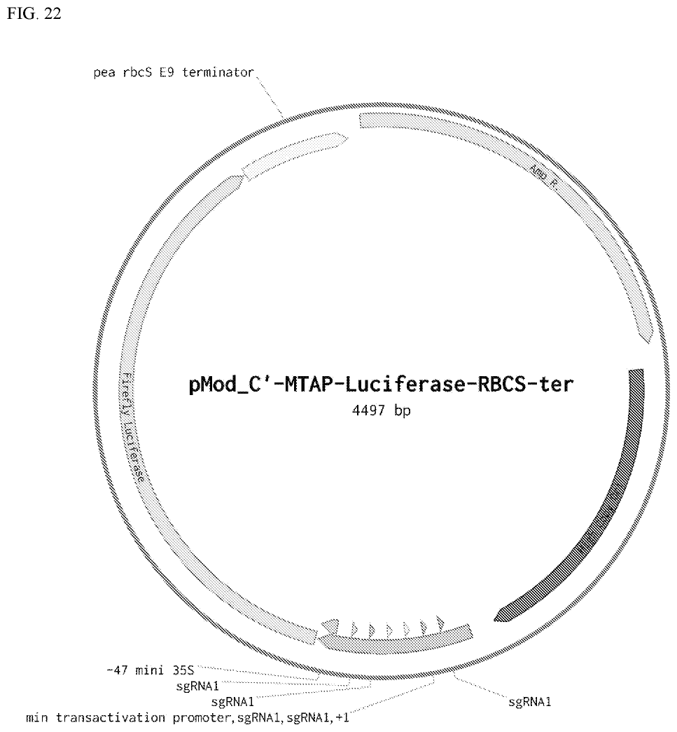

All MoonTag variations designed were tested for their ability to activate a promoter driving a luciferase reporter in a Setaria protoplast transient expression system (FIG. 3, FIG. 22). Promoter activation, as determined by luciferase activity, was observed in the presence of all MoonTag constructs tested, albeit to different levels (FIG. 4A). Luciferase activity was the highest with MoonTag that included the NbGP41-VP64-GB1 component (MT2). The lowest activity was observed with MoonTag that included the NbGP41-sfGFP-VP64-GB1 component (MT1). Increasing the copy number of the GP41 amino acid binding sequence (SEQ ID NO:1) on the binding polypeptide fused to dCas9 lead to higher activation levels but not in the MoonTag version that includes the NbGP41-VP64-GB1 component. The MoonTag system seems to provide similar or slightly better activation levels than the SunTag system (FIG. 4A). These indicate that MoonTag activating system works as a transcriptional activator capable of inducing expression of a reporter gene at similar levels as the SunTag activating system.

The ability of the MoonTag activation system to activate endogenous genes in protoplasts was tested. sgRNAs (Table 1) target MoonTag to the promoter of four endogenous Setaria genes, WUSCHEL, CLAVATA3, MYB21, and CSP4 were designed. Transient expression of the components of the MoonTag system in the presence of sgRNAs (Table 1) binding to the promoter of endogenous genes lead to the increased expression of all targets (FIG. 4B). The large increase in activation were observed for WUSCHEL, CLAVATA3 and MYB21. Modest levels of activation were observed for CSP4 (FIG. 4B). Taken together these observations indicate that the MoonTag system is an efficient activator of transgenes and endogenous genes when transiently expressed in Setaria protoplasts.

TABLE 1
MoonTag sgRNAs targeting various promoters of Setaria genes

| | | Promoter |
|---|---|---|
| sgRNA name | SEQ ID NO: | Target |
| SvWUSCHEL_sgRNA7.9 | 8 | WUSCHEL |
| SvWUSCHEL_sgRNA7.10 | 9 | WUSCHEL |
| SvWUSCHEL_sgRNA7.12 | 10 | WUSCHEL |
| SvCLAVATA3_sgRNA2 | 11 | CLAVATA3 |
| SvCLAVATA3_sgRNA3 | 12 | CLAVATA3 |
| SvCLAVATA3_sgRNA4 | 13 | CLAVATA3 |
| SvMYB21_sgRNA7 | 14 | MYB21 |
| SvMYB21_sgRNA9 | 15 | MYB21 |
| SvMYB21_sgRNA11 | 16 | MYB21 |
| SvCSP4_sgRNA17.2 | 17 | CSP4 |
| SvCSP4_sgRNA17.3 | 18 | CSP4 |
| SvCSP4_sgRNA17.6 | 19 | CSP4 |

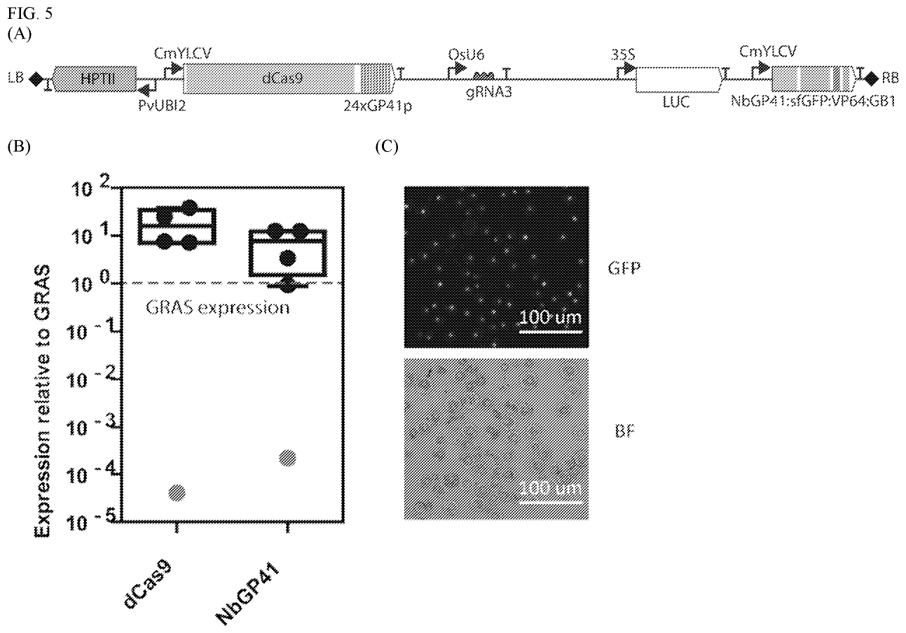

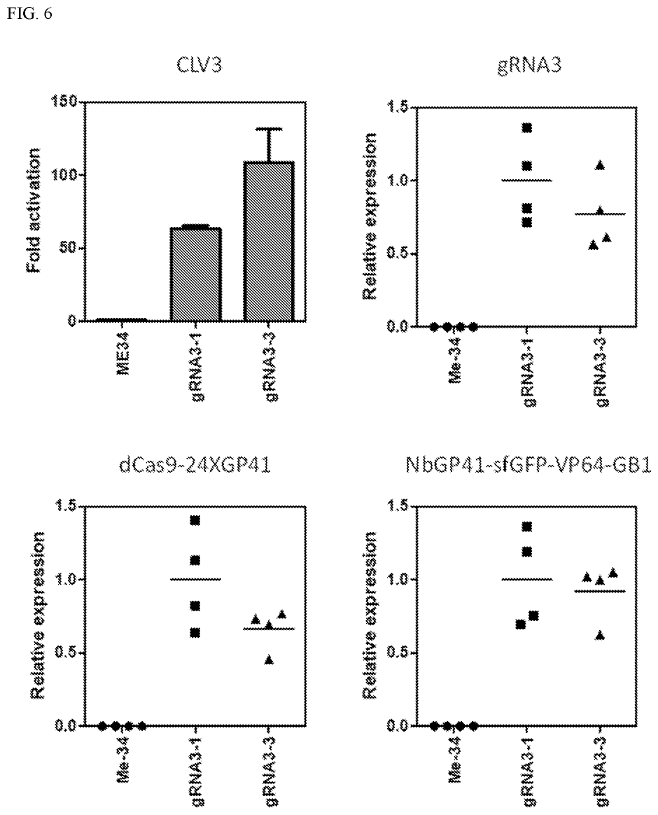

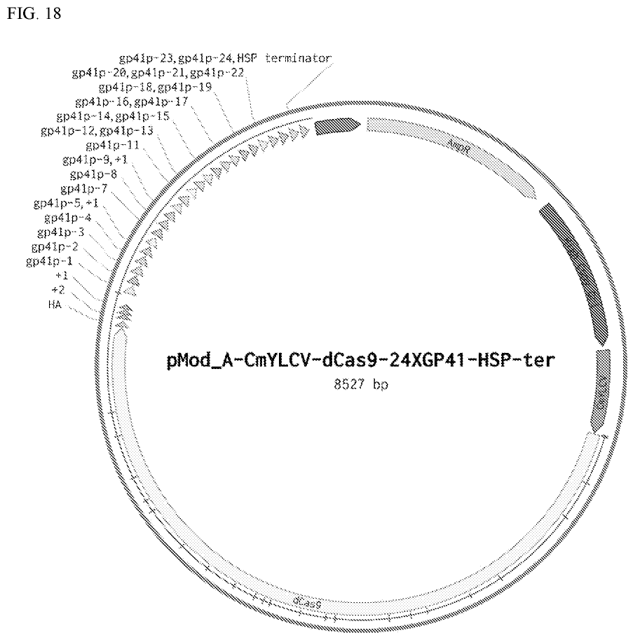

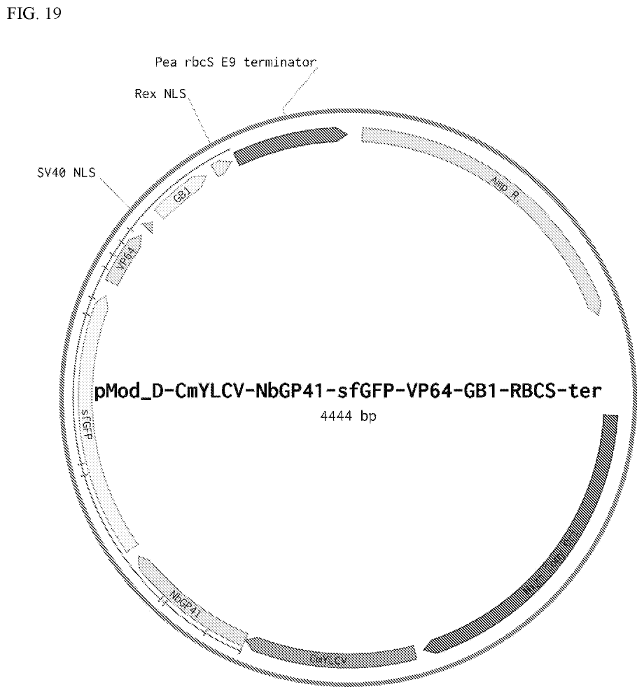

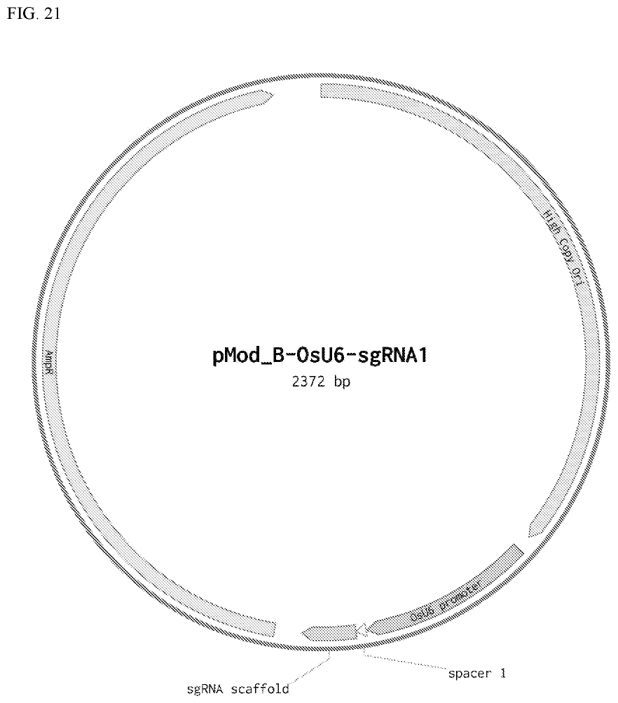

The activity of the MoonTag system was investigated in Setaria plants by stably expressing the components of the MoonTag system. Because Setaria can be transformed using Agrobacterium, a binary vector (FIG. 18, FIG. 19, and FIG. 21) was constructed that can express MoonTag with a sgRNA that targets the promoter of the CLV3 gene. This construct included dCas9-24XGP41 and NbGP41-sfGFP-VP64-GB1 driven by the cm YLCV promoter. The construct also included the CLV3-sgRNA driven by a rice U6 pol III promoter. Additionally, the construct included a HPTII selectable marker conferring resistance to hygromycin (FIG. 5A). After Agrobacterium mediated transformation, several hygromycin resistant plants were obtained and transferred to soil. Plants transferred to soil did not show any growth defects or obvious morphological changes. The expression of dCas9-24XGP41 and NbGP41-sfGFP-VP64-GB1 in four of these lines was measured. All the plants expressed the MoonTag components at higher levels than the GRAS endogenous control (FIG. 5B). A GFP signal from the expression of NbGP41-sfGFP-VP64-GB1 was observed in the nuclei of protoplast derived from leaves of MoonTag expressing plants (FIG. 5C). Three of the plants analyzed also showed increased expression levels of the CLV3 target gene. Two transgenic lines showing the highest levels of CLV3 activation were then grow for two generations to produce homozygous seeds. Expression of CLV3 in homozygous lines MoonTag lines was shown to be between 50-fold to 100-fold higher than CLV3 in the wild-type ME34 control (FIG. 6). Expression of the MoonTag components (dCas9-24XGP41 and NbGP41-sfGFP-VP64-GB1) and the sgRNA were also maintained in the homozygous lines (FIG. 6). These observations suggest that Setaria transgenic plants stably expressing high levels of MoonTag components can be obtained without deleterious effects. More importantly, transgenic plants expressing the MoonTag components targeted to the CLV3 promoter by a single sgRNA resulted in the increased expression of this gene in Setaria.

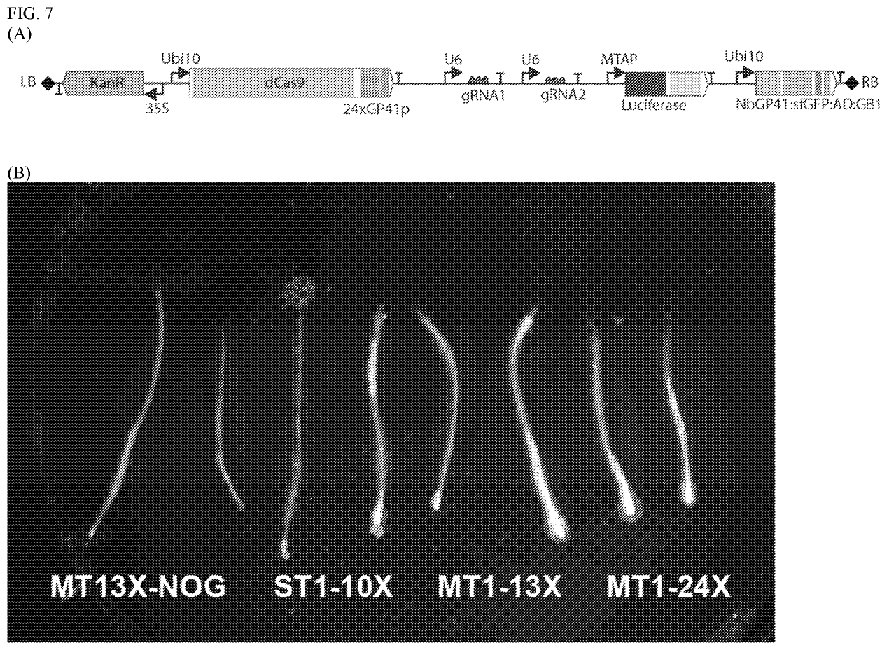

The ability of the MoonTag system to activate genes in eudicotyledonous species such as tomato and Arabidopsis was studied. In tomato, hairy roots produced by Agrobacterium rhizogenes were used to test the MoonTag and SunTag activator systems. The constructs used included a luciferase reporter driven by a promoter that was activated by either the MoonTag system or the SunTag system. As a control, a construct expressing all components of the MoonTag system except the targeting sgRNA was generated. After transformation, the hairy roots obtained with the different constructs were analyzed for expression of the luciferase reporter and expression of the activator system components. Expression analysis in hairy roots indicate that both activator systems express the luciferase reporter to higher levels than roots expressing MoonTag without a sgRNA. Similarly, hairy roots treated with D-luciferin clearly showed increased luminescent signal in plants transformed with the MoonTag system and the SunTag system when compared to that of the control roots expressing the MoonTag system without a sgRNA. Luciferase expression in roots transformed with the MoonTag system was more efficient than luciferase expression obtained with the SunTag system (FIG. 7). However, the SunTag system used included ten copies of the GCN4 amino acid binding sequence on the binding peptide whereas the MoonTag systems had either 13 or 24 copies of the GP41 amino acid binding sequence (SEQ ID NO:1) on the binding peptide. Thus, the MoonTag system appears to be at least as efficient, if not better, as the SunTag system in the activation of a reporter gene in tomato.

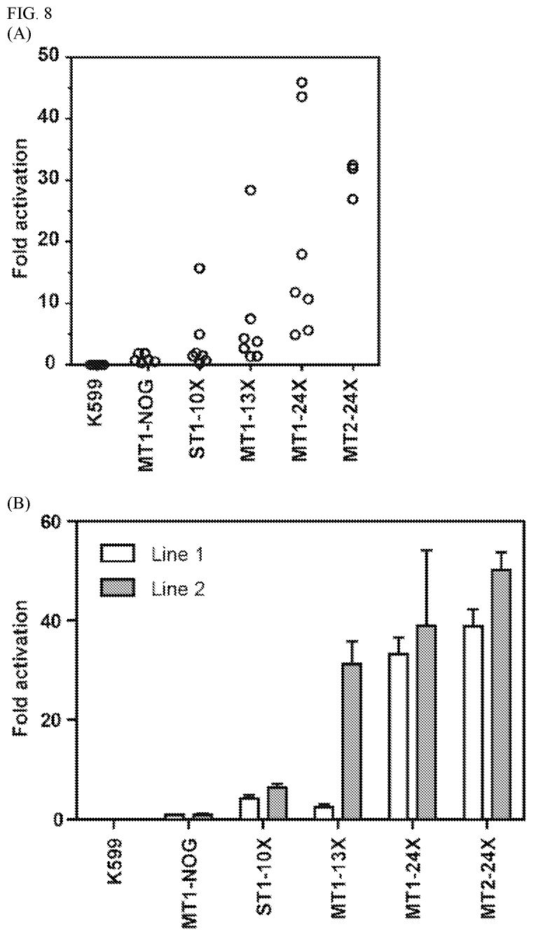

Expression analysis revealed no major differences in the expression of the components of both the SunTag and MoonTag activator systems (FIG. 8A). However, the GFP signal of the nanobody component of the MoonTag system (NbGP41-sfGFP-VP64-GB1) is usually higher than the antibody component of the SunTag system (ScFv-sfGFP-VP64-GB1) (FIG. 8B). This suggests that even though the nanobody component of the MoonTag system and the antibody component of the SunTag system are expressed at the same level, the nanobody content is higher than the antibody content.

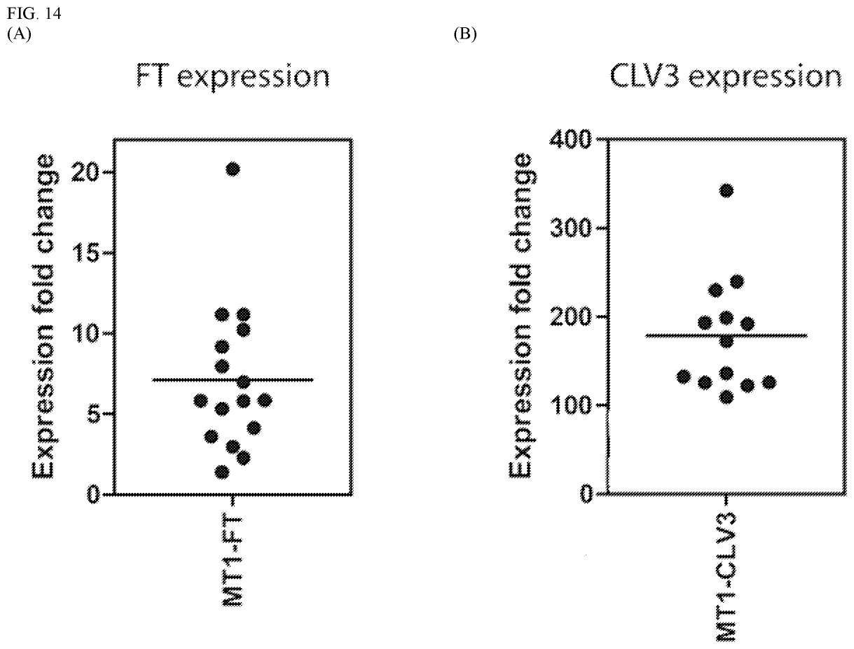

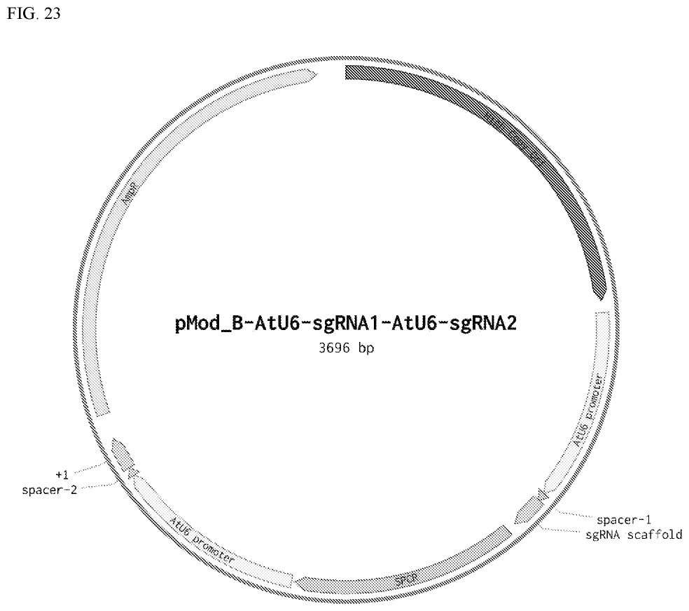

Transgenic plants expressing the MoonTag system with various sgRNAs (Table 2) that targeted the CLAVATA3 (CLV3) and FLOWERING LOCUS T (FT) genes were generated in Arabidopsis. For transformation with Agrobacterium, each construct contained the MoonTag system components driven by the UBI10 constitutive promoter. The construct also included two sgRNAs targeting the promoter of CLV3 or FT (e.g., FIG. 23). Additionally, the construct included a selectable marker conferring kanamycin resistance. After transformation, kanamycin resistant seedlings expressing the MoonTag system targeting the CLV3 gene (13 lines) and the FT (16 lines) gene were obtained. Expression of CLV3 in the lines targeting the CLV3 gene was between 200-fold to 400-fold higher that in the wild type system (FIG. 14B). Expression of FT in the lines targeting FT ranged between two- to 20-fold higher than that of the wild type (FIG. 14A). Thus, like in Setaria and tomato, the MoonTag system can induce high expression of its target genes in Arabidopsis.

TABLE 2
sgRNAs targeting the CLAVATA3 (CLV3)
and FLOWERING LOCUS T (FT) genes

| sgRNA name | SEQ ID NO: | Promoter Target |
|---|---|---|
| AtCLV3_sgRNA1 | 20 | CLV3 |
| AtCLV3_sgRNA2 | 21 | CLV3 |
| AtFT_sgRNA-A | 22 | FT |
| AtFT_sgRNA-B | 23 | FT |

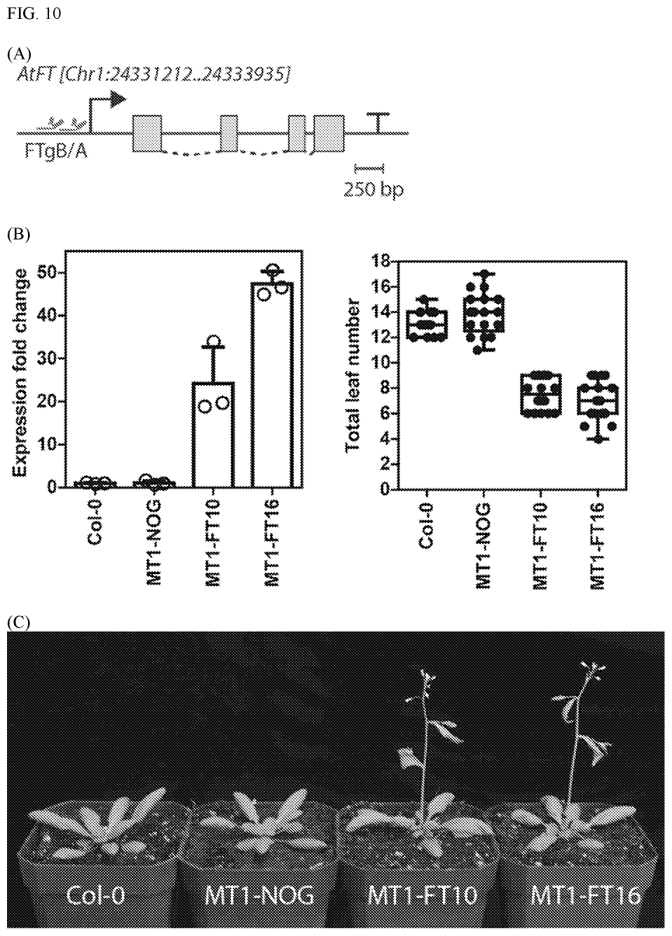

Different activator domains were experimented with in the MoonTag system. For example, the activator domain VP64 was replaced by novel activation domains (ADs) previously identified as improving the efficiency of the SunTag system. Different versions of the MoonTag system, each carrying a different activation domain (AD1-AD6, FIG. 10), were generated and tested for their ability to activate transcription of a luciferase reporter in a Setaria protoplast transient system. Three systems increase the transcriptional activation provided by MoonTag system (AD4, FIG. 15). Thus, the activation efficiency of MoonTag could be significantly improved when combined with more efficient activation domains.

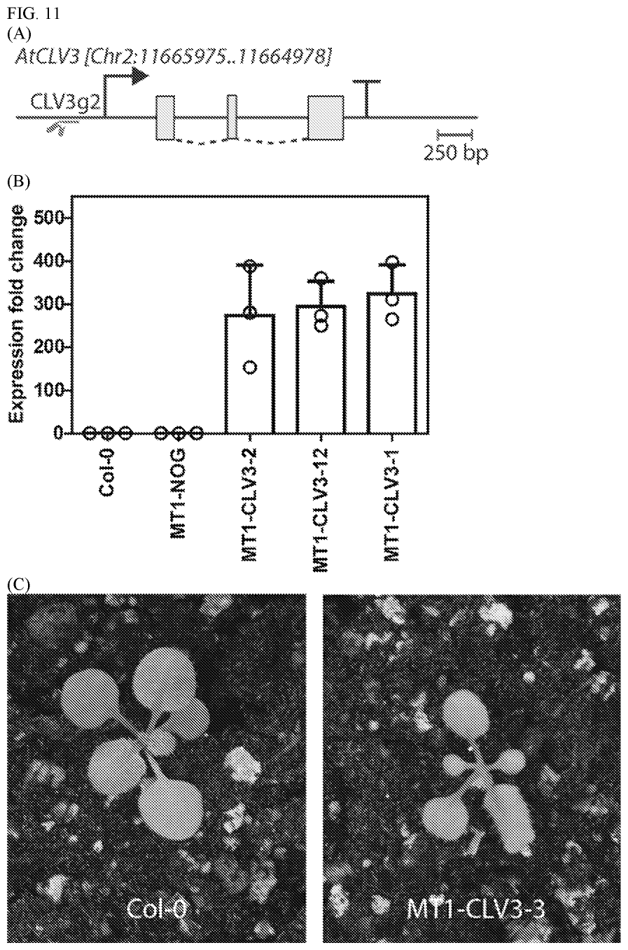

FIG. 10A illustrates an exemplary embodiment of the FT gene showing the position of the gRNAs designed to activate FT in relation to the TSS. FIG. 11A illustrates an exemplary embodiment of the CLV3 gene showing the position of the gRNAs designed to activate CLV3. RT-qPCR expression of the FT gene in the indicated transgenic lines is shown in FIG. 10B (left, while RT-qPCR expression of the CLV3 in the indicated lines is shown in FIG. 11B (left). The right panel of FIG. 10B shows the total rosette leaf number until flowering shown by each transgenic line. FIG. 10C and FIG. 11C show the phenotypes generated by the overexpression of FT (FIG. 10C) and the overexpression of CLV3 (FIG. 11C).

These results demonstrate that the level of overexpression achievable via MoonTag programmable transcription activators is capable of driving phenotypes in transgenic plants. The exemplary embodiments demonstrate two separate phenotypes: early flowering by overexpressing FT and small size/loss of meristem cell by overexpressing CLV3. These results demonstrate that driving overexpression of gene targets results in physiological differences in plant traits that are readily observable at the whole-plant level.

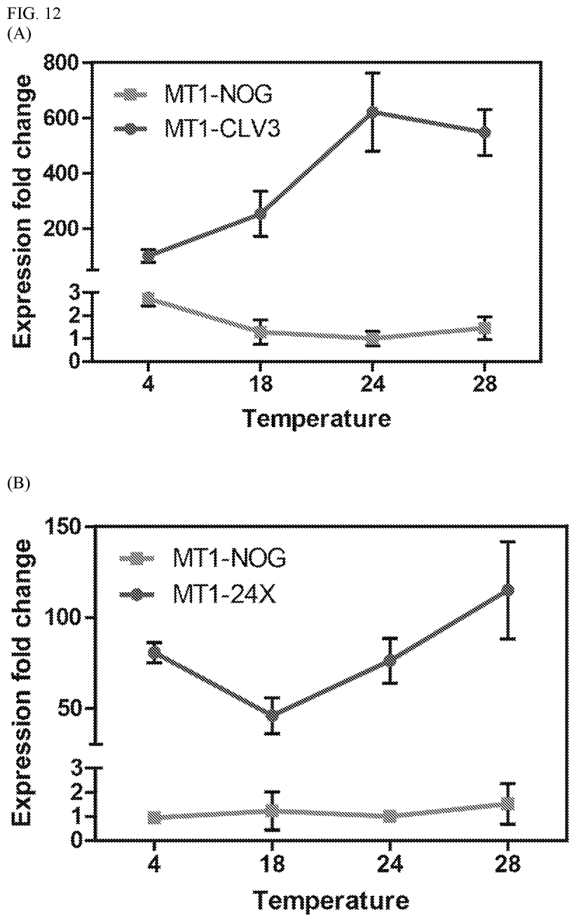

MoonTag activation is reduced at lower temperature in Arabidopsis and tomato. FIG. 12A shows activation of CLV3 by MoonTag in Arabidopsis seedlings incubated at different temperatures. FIG. 12B shows luciferase expression in hairy roots incubated at different temperatures. These results demonstrate that robust overexpression with MoonTag programmable transcription activators is achievable in diverse environments/temperatures. While Cas9 nuclease activity dramatically drops at temperatures below 25° C., dCas9-based activators retain the ability to drive gene expression at temperatures as low as 4° C. These results suggest that traits engineered using MoonTags will function in diverse environmental conditions.

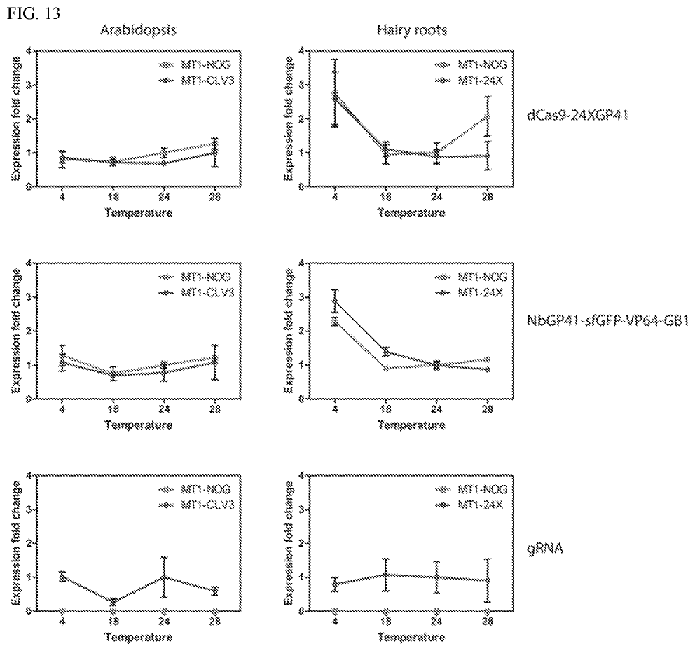

To further examine MoonTag-driven expression at different temperatures, expression of each individual component of the MoonTag system was examined. FIG. 13 shows the expression of MoonTag components (dCas9, top; co-activator, middle; gRNA, bottom) at different temperatures. Expression was normalized to that of TUB2 for Arabidopsis (left) and to Actin2 for tomato hairy roots (right). These results support the data in FIG. 12 showing that the components of the MoonTag PTA are expressed equally well across the temperature range tested.

### Synthetic Promoters

In another aspect, the present disclosure describes synthetic promoters. Generally, the synthetic promoter includes a core promoter and a trans-activation region (TA). The core promoter includes the region immediately upstream and/or immediately downstream of the transcriptional initiation site (TSS). The TA region is upstream the TSS. The TA region includes at least one binding sequence for a transcription factor to bind. Binding of the transcription factor promotes transcription of a target gene or represses transcription of the target gene.

The core promoter generally includes the regions of the DNA near the TSS where the transcription pre-initiation complex and RNA pol II bind. Example core promoter motifs include, but are not limited to, TATA box, BRE, DPE, MTE, DCE, and XCPE1. Core promoter elements may be adapted from core promoters found in eukaryotes or prokaryotes. For example, core promoter elements may be adapted from core promoters in Arabidopsis.

The core promoter may include DNA sequences downstream, upstream, or both downstream and upstream to the TSS. The core promoter may include a DNA sequence that is upstream of the TSS. In one or more embodiments, the core promoter includes multiple DNA sequences that are upstream of the TSS. The core promoter may include a DNA sequence that is downstream of the TSS. In one or more embodiments, the core promoter includes multiple DNA sequences downstream of the TSS. In one or more embodiments, the core promoter includes a DNA sequence upstream the TSS and a DNA sequence downstream the TSS. In one or more embodiments, the core promoter includes multiple DNA sequences upstream the TSS and a DNA sequence downstream the TSS. In one or more embodiments, the core promoter includes a DNA sequence upstream the TSS and multiple DNA sequences downstream the TSS. In one or more embodiments, the core promoter includes multiple DNA sequences upstream the TSS and multiple DNA sequences downstream the TSS.

The location of the core promoter may vary. In one or more embodiments, the core promoter is located 70-90 base pairs upstream the TSS. In one or more embodiments, the core promoter is located at 5-24 base pairs downstream the TSS and 5-30 base pairs upstream the TSS. In one or more embodiments, the core promoter is located 15-20 base pairs downstream the TSS. In one or more embodiments, the core promoter is located 80-85 base pairs upstream the TSS. In one or more embodiments, the core promoted is located 15-20 base pairs upstream the TSS and 80-85 base pairs downstream the TSS.

Generally, the TA region includes at least one binding sequence recognized by transcription factors. The recognition of transcription factors to the TA region either promotes or represses gene transcription. In the present disclosure, transcription factors are recruited to the gene of interest through binding of the sgRNA of a CRISPR-Cas-based transcriptional activator system to a sgRNA binding sequence in the TA. For Example, activation domains may be recruited to the TA region through binding of the sgRNA of the MoonTag system.

The location of the TA region relative to the TSS may vary. In the present disclosure, the location of the TA region is described by the position of the nucleotide in the TA region that is the closest to the TSS. The TA region extends upstream from the position of the nucleotide that is closest to the TSS. In one or more embodiments the location of the TA region located at least 70 base pairs, at least 80 base pairs, at least 90 base pairs, at least 100 base pairs, or at least 150 base pairs upstream from the TSS. In one or more embodiments, the TA region is located no greater than 200 base pairs, no greater than 150 base pairs, no greater than 100 base pairs, no greater than 90 base pairs, no greater than 80 base pairs, or no greater than 70 base pairs upstream from the TSS. In one or more embodiments, the TA region is located from 70 to 200 base pairs, 70 to 150 base pairs, 70 to 100 base pairs, 70 to 90 base pairs, or 70 to 80 base pairs upstream the TSS. In one or more embodiments, the TA region is located from 80 to 200 base pairs, 80 to 150 base pairs, 80 to 100 base pairs, or 80 to 90 base pairs upstream the TSS. In one or more embodiments, the TA region is located from 90 to 200 base pairs, 90 to 150 base pairs, or 90 to 100 base pairs upstream the TSS. In one or more embodiments, the TA region is located from 100 to 200 base pair or 100 to 150 base pairs upstream the TSS. In one or more embodiments, the TA region is located from 150 to 200 base pairs upstream the TSS.

The TA region may have one or more sgRNA binding sequences. In one or more embodiments, the TA has at least one, at least two, at least three, at least four, at least five, at least six, at least seven, or at least ten sgRNA binding sequences. In one or more embodiments, the TA has no greater than 15, no greater than ten, no greater than seven, no greater than six, no greater than five, no greater than four, no greater than three, no greater than two, or no greater than one sgRNA binding sequences. In one or more embodiments, the TA has one to 15, one to ten, one to seven, one to six, one to five, one to four, one to three, or one to two sgRNA binding sequences. In one or more embodiments, the TA has two to 15, two to ten, two to seven, two to six, two to five, two to four, or two to three sgRNA binding sequences. In one or more embodiments, the TA has three to 15, three to ten, three to seven, three to six, three to five, or three to four sgRNA binding sequences. In one or more embodiments, the TA has four to 15, four to ten, four to seven, four to six, or four to five sgRNA binding sequences. In one or more embodiments, the TA has five to 15, five to ten, five to seven, or five sgRNA binding sequences. In one or more embodiments, the TA has six to 15, six to ten, or six to seven sgRNA binding sequences. In one or more embodiments, the TA has seven to 15 or seven to ten sgRNA binding sequences. In one or more embodiments, the TA has ten to 15 sgRNA binding sequences.

In one or more embodiments when the TA region includes more than one sgRNA binding sequence, the sgRNA binding sequences may all be the same. In one or more embodiments when the TA region includes more than one sgRNA binding sequence, the sgRNA binding sequences may all be different same. In one or more embodiments when the TA region includes three or more sgRNA binding sequence, and least two sgRNA binding sequences are the same and at least one sgRNA binding sequence is different.

When the TA region includes more than one sgRNA binding sequence, a sgRNA binding sequence can be separated from a neighboring sgRNA sequence by a sgRNA spacer sequence. In one or more embodiments, the sgRNA spacer is at least 20 base pairs, at least 30 base pairs, at least 40 base pairs, at least 50 base pairs, or at least 60 base pairs in length. In one or more embodiments, the sgRNA spacer sequence is no greater than 100 base pairs, no greater than 60 base pairs, no greater than 50 base pairs, no greater than 40 base pairs, no greater than 30 base pairs, or no greater than 20 base pairs in length. In one or more embodiments, the sgRNA spacer sequence is 20 to 100 base pairs, 20 to 60 base pairs, 20 to 50 base pairs, 20 to 40 base pairs, or 20 to 30 base pairs in length. In one or more embodiments, the sgRNA spacer sequence is 30 to 100 base pairs, 30 to 60 base pairs, 30 to 50 base pairs, or 30 to 40 base pairs in length. In one or more embodiments, the sgRNA spacer sequence is 40 to 100 base pairs, 40 to 60 base pairs, or 40 to 50 base pairs in length. In one or more embodiments, the sgRNA spacer sequence is 50 to 100 base pairs, or 50 to 60 base pairs in length. In one or more embodiments, the sgRNA spacer sequence is 60 to 100 base pairs in length. In one or more embodiments, the sgRNA spacer is 30 to 50 base pairs in length. In one or more embodiments the sgRNA spacer is 38 base pairs in length.

In one or more embodiments when the sgRNA includes more than one sgRNA spacer sequence the sgRNA spacer sequences may all be the same. In one or more embodiments when the TA region includes more than one sgRNA spacer sequence, the sgRNA spacer sequences may all be different. In one or more embodiments when the TA region includes three or more sgRNA spacer sequence, at least two sgRNA spacer sequences are the same and at least one sgRNA spacing sequence is different.

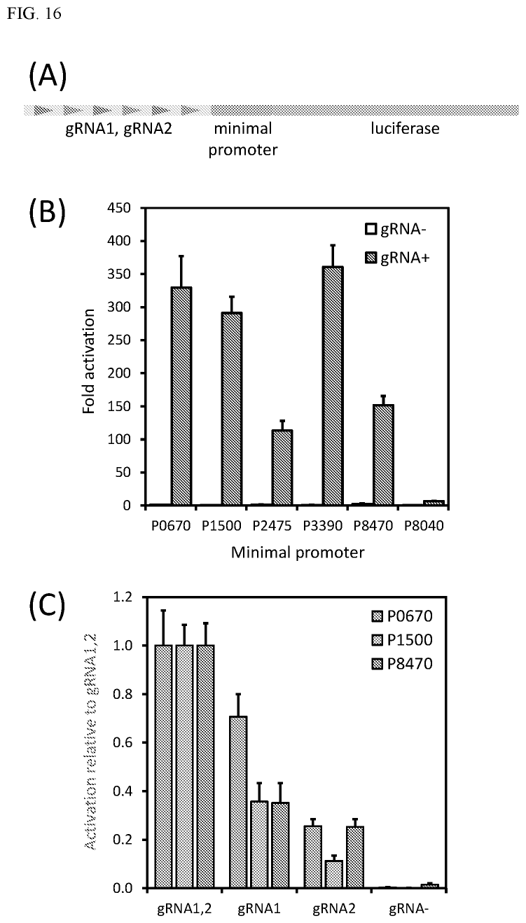

Turning to FIG. 16, a modular approach was used for the design of synthetic plant promoters. This approach divides a promoter into a minimal or core promoter. The core promoter includes the location where the transcription pre-initiation complex and RNA pol II binds. The synthetic promoter also includes a trans-activation (TA) region upstream of the TSS. The trans-activation region includes binding sequences for transcription factors that stimulate or repress transcription (FIG. 16A). The region between 15-20 bp downstream of the TSS and the region between 80-85 bp upstream of the TSS was chosen for the core promoter. Sequences of the core promoters (Table 3) were obtained from six Arabidopsis promoters chosen at random from 576 plant promoters with experimentally verified transcription initiation sites present in the PlantProm database (Shahmuradov et al., Nucleic Acid Res. (2003) 31, 114-117).

TABLE 3
Arabidopsis promoters

| Promoter Name | SEQ ID NO: |
|---|---|
| P0670 | 27 |
| P1500 | 28 |
| P2475 | 29 |
| P3390 | 30 |
| P8470 | 31 |
| P8040 | 32 |

The TA region was designed to contain six sgRNA binding sites (three for sgRNA1 (SEQ ID NO: 6) and three for sgRNA2 (SEQ ID NO:7)) separated by 38 bp. Because the binding of the CRISPR activator domain depends on the spacer sequence and the protospacer adjustment motif (PAM) region, sequence diversity was created by randomizing the sequence that separates the sgRNA binding sites. This allows for the creation of TA regions with less than ˜20% of duplicated sequences without losing the activation strength provided by the presence of the sgRNAs. When the TA regions are assembled with different minimal promoters, synthetic promoters are obtained that share a minimal amount of duplicated sequences but could drive the expression of their coding sequences at the same levels.

Three TA regions (TA-1, SEQ ID NO:24; TA-2, SEQ ID NO:25; and TA-3, SEQ ID NO: 26) were created. The sequences of TA-1 (SEQ ID NO:24), TA-2 (SEQ ID NO:25), and TA-3 (SEQ ID NO:26) are diversified to the point where none could not be recognized as similar to another by blast comparison. However, all still contain the six sgRNA binding sites. TA-1 (SEQ ID NO: 24) was used to assemble six synthetic promoters to drive luciferase (Luc) expression (FIG. 16A). When these constructs were transiently expressed together with the MoonTag activator with sgRNA1 (SEQ ID NO:6) and sgRNA2 (SEQ ID NO:7) in Setaria protoplasts, at least three of promoters provided similar luciferase activity, two showed low activity, and one of them was barely above background (FIG. 16B). In contrast, very low Luciferase activity was observed when the MoonTag activator was transformed without sgRNAs. This data suggest that synthetic promoters activated by the MoonTag system could be obtained by a modular approach.

The activity of the promoters was compared when transformed with the MoonTag activator and either sgRNA1 (SEQ ID NO:6), sgRNA2 (SEQ ID NO:7) or both. This study mimics the activation levels produced when the promoters are activated by the binding in three or six of the sgRNA binding sites. Lower activation levels were observed when the promoters are bound at only three sgRNA binding sites than when six are bound. Furthermore, activation levels with sgRNA1 (SEQ ID NO:6) only are higher than with sgRNA2 (SEQ ID NO:7) only (FIG. 11C). Together these data suggest that the activation strength of the promoter is tunable by changing the number and position of the sgRNA binding sites.

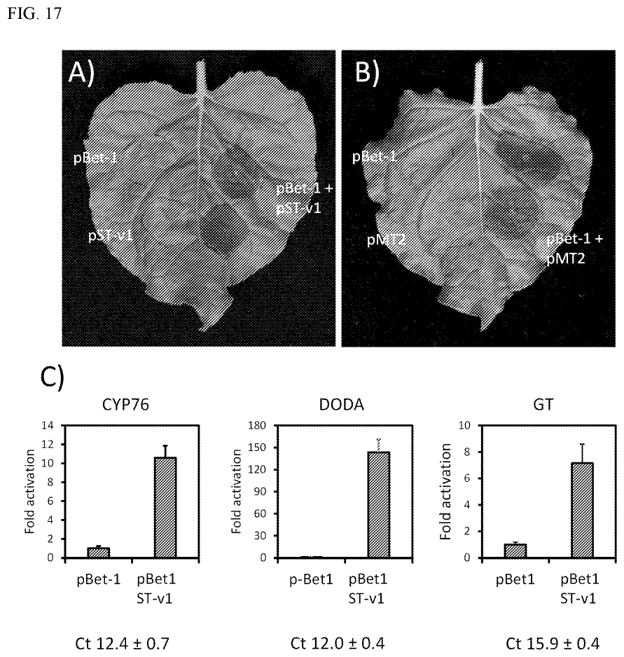

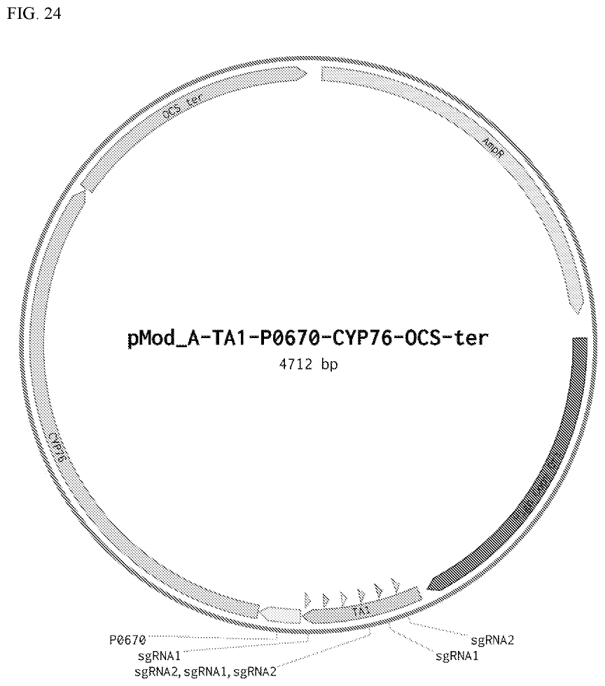

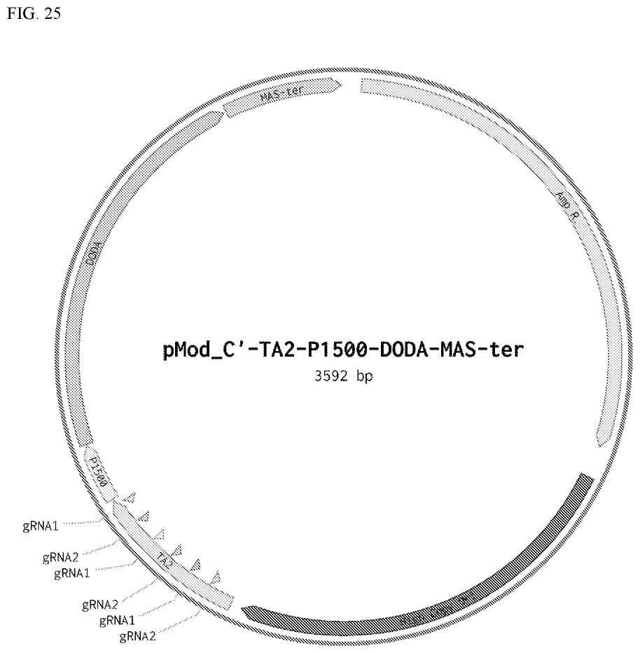

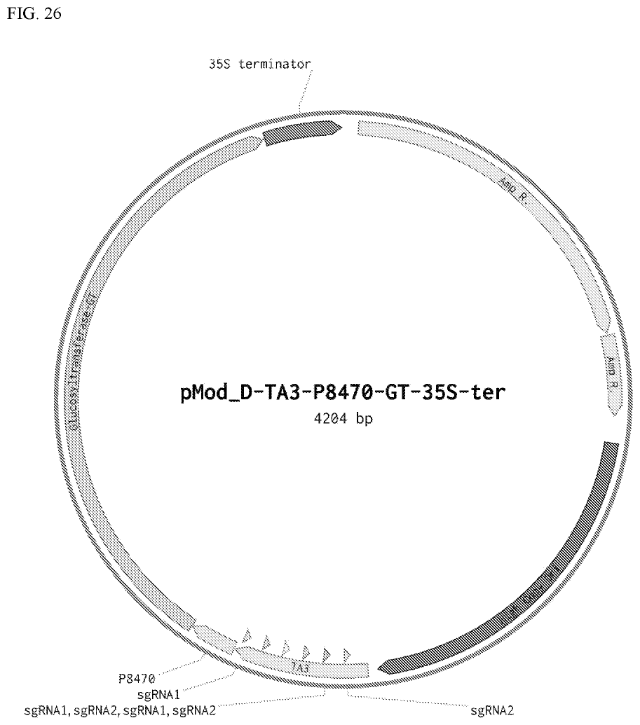

To test the ability of the synthetic promoters and the CRISPR-Cas activators to induce expression of multiple genes the Betalain pathway was used as a test case. Biosynthesis of betalains requires three enzymes (CYP76AD1, DODA, and glucosyltransferase (GT)) to convert tyrosine into betalain. Betalain is a bright red color compound seen in beets, dragon fruit and other plants. Three different synthetic promoters were assembled by combining TA-1, TA-2, and TA-3, with three different core promoters. The resulting promoters TAP-1 (FIG. 24), TAP-2 (FIG. 25) and TAP-3 (FIG. 26) were placed upstream of the coding sequences of CYP76AD1, DODA, and GT, respectively (see Table 4). These units were further assembled with sgRNA1 (SEQ ID NO:6) and sgRNA2 (SEQ ID NO:7) expression cassettes into a single binary vector (pBet-1). A binary vector containing the SunTag system activator components driven by the Arabidopsis UBI10 constitutive promoter was created and named ST-v1. A binary vector containing the MoonTag system activator components driven by the Arabidopsis UBI10 constitutive promoters was created and named pMT2. The binary vectors were transformed into Agrobacterium tumefaciens and the resulting strains used to transiently express the genes in Nicotiana benthamiana leaves by agroinfiltration. Expression of the betalain biosynthesis genes driven by the synthetic promoters and sgRNA1 (SEQ ID NO:6) and sgRNA2 (SEQ ID NO:7) by themselves (pBet-1) did not result in any betalain accumulation, suggesting the synthetic promoters are inactive (FIG. 17A, FIG. 17B). Similarly, expression of the SunTag system activator (FIG. 17A) and MoonTag system activators (FIG. 17B) by themselves did not produce any betalain. However, co-expression of the betalain genes with the SunTag activator led to accumulation of betalains as demonstrated by the patch of red pigment formed in the agroinfiltration site (FIG. 17A). Co-expression of pBet-1 with the MoonTag activator led to accumulation of betalains as demonstrated by the patch of red pigment formed in the agroinfiltration site (FIG. 17B). RT-qPCR analysis of the expression of CYP76AD1, DODA, and GT confirm the induction expression of the three genes in the presence of the SunTag activator (FIG. 17C). This same analysis indicates that the promoter for CYP76AD1 and GT show some background expression whereas the DODA is not expressed at all in the absence of the SunTag activator. This data indicates that the synthetic promoters and the CRISPR-Cas activators could be used for the coordinated expression of multiple genes. Thus, by regulating the expression of the activator with tissue-specific or inducible promoters it should be possible to control the expression of the genes under control of the synthetic promoters.

TABLE 4
Components of TAP synthetic promoters

| TA | Core promoter | Core promoter | TA | |
|---|---|---|---|---|
| name | name | SEQ ID NO: | name | SEQ ID NO: |
| TAP-1 | P0670 | 27 | TA1 | 24 |
| TAP-2 | P1500 | 28 | TA2 | 25 |
| TAP-3 | P8470 | 31 | TA3 | 26 |

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The term “polypeptide” refers to a sequence of amino acid residues without regard to the length of the sequence. Therefore, the term “polypeptide” refers to any amino acid sequence having at least two amino acids and includes full-length proteins, fragments thereof, and/or, as the case may be, polyproteins.

The term “protein” refers to any sequence of two or more amino acid residues without regard to the length of the sequence, as well as any complex of two or more separately translated amino acid sequences. Protein also refers to amino acid sequences chemically modified to include a carbohydrate, a lipid, a nucleotide sequence, or any combination of carbohydrates, lipids, and/or nucleotide sequences. As used herein, “protein,” “peptide,” and “polypeptide” are used interchangeably.

The term “antibody” refers to a molecule that contains at least one antigen binding site that immunospecifically binds to a particular antigen target of interest. The term “antibody” thus includes but is not limited to a full length antibody and/or its variants, a fragment thereof, peptibodies and variants thereof, monoclonal antibodies (including full-length monoclonal antibodies), polyclonal antibodies, multispecific antibodies (e.g., bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. Thus, as used herein, the term “antibody” encompasses antibody fragments capable of binding to a biological molecule (such as an antigen or receptor) or a portion thereof, including but not limited to Fab, Fab′ and F(ab′) 2, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments. The antibody can be of any type (e.g., IgG, IgE, IgM, IgD, IgA and IgY), class (e.g., IgG1, IgG2, IgG3, IgG4, IgA1 and IgA2), or subclass. The present invention is illustrated by the following examples. It is to be understood that the particular examples, materials, amounts, and procedures are to be interpreted broadly in accordance with the scope and spirit of the invention as set forth herein.

### EXAMPLES

### Construction of the MoonTag Activator

The Dead Cas9 Coding Sequence (dCas9; SEQ ID NO:5) was Obtained from pEG302 22Aa SunTag VP64 nog (plasmid #120251; Addgene, Watertown, MA). DNA fragments containing different copy numbers of the GP41 peptide (SEQ ID NO: 1) separated by GS linker were derived from the plasmid 24×MoonTag-kif18b-24xPP7 (plasmid #128604; Addgene, Watertown, MA). The DNA fragment encoding the GP41 nanobody (SEQ ID NO:2) was cloned from the plasmid Nb-gp41-GFP (MoonTag-Nb-GFP) (plasmid #128602; Addgene, Watertown, MA).

### Protoplast Isolation and Transfection

Protoplasts from Setaria leaves were isolated as described before (Weiss et al., Plant J., 2020, 104:828-838). Transfection was carried out using the polyethylene glycol (PEG)-mediated method. For transfection of protoplasts for RNA analysis, 500,000 cells were mixed with plasmid DNA corresponding to the different constructs (10 μg per construct) in 20% PEG for 10 minutes. After transfection protoplasts incubated at room temperature in the dark for 16 to 18 hours. Protoplast transfection for luciferase assays was carried out with 100,000 cells and 2 ug of plasmid DNA for each construct. A plasmid expressing Renilla luciferase from an SWS promoter was added to be used in downstream analysis to normalize the activity of firefly luciferase.

### Luciferase Assay of Protoplasts

Protoplasts were collected by centrifugation, resuspended in 20 ul of passive lysis buffer (Promega, Madison WI) and lysis allow to happen for 15 minutes at room temperature with shaking at 40 rpm. Firefly and Renilla Luciferase activities in the lysate were then determined with a Dual-Luciferase Reporter Assay System (Promega, Madison WI) and a GIOMAX explorer plate reader (Promega, Madison WI) following the manufacturer instructions. Firefly luciferase activity in the different treatments was normalized to that of Renilla Luciferase.

### RNA Isolation

RNA was isolated from the different plant tissues using the Trizol reagent (Thermo Fischer Scientific, Waltman MA) following the manufacturer instructions. For Setaria, Arabidopsis and tomato hairy roots 50-100 mg of tissue were extracted 1 ml of Trizol whereas for Setaria protoplasts, only 750 ul of Trizol was used for 500000 cells. RNA was resuspended in nuclease-free water (TAKARA) and then treated with DNAse using the TURBO DNA-Free™ Kit (Invitrogen, Thermo Fisher Scientific, Inc., Waltham, MA).

### Quantitative Reverse Transcription PCR (RT-qPCR)

RT-qPCR was carried out from the isolated RNA using the Luna Universal One-Step RT-qPCR Kit (New England Biolabs, Inc., Ipswich, MA) following the manufacturer instructions. RT-qPCR for RNA samples from Setaria leaves, Arabidopsis seedlings and tomato hairy roots was done using 100-150 ng of mRNA per reaction. For protoplasts, each reaction was performed using 25-35 ng of RNA. Expression of the genes tested in Setaria, tomato hairy roots and Arabidopsis were normalized to that of GRAS, ACT2, and TUBULIN 2, respectively.

Nicotiana benthamiana Agroinfiltration

Transient expression of binary vectors into Nicotiana benthamiana by Agrobacterium infiltration of leaves was carried out according to (Sparkes et al., Nat Protoc (2006) 1, 2019-2025). Agrobacterium strains carrying the binary vector pBet-1 and pST-v1 were growth separately and mixed in a 1:1 ratio right before transformation.

### Arabidopsis Transformation

Arabidopsis plants ecotype Columbia-0 was transformed with Agrobacterium tumefaciens carrying the binary vectors of interest and using the “floral dip” method (Clough and Bent, Plant J. (1998) 16, 735-743). After transformation seeds were harvested and putative transformants were identifying by kanamycin selection in MS media (Murashige and Skoog medium) containing 50 mg/L of kanamycin. Seedlings resistant to kanamycin were transferred to soil and grown until the plants set seeds.

### Temperature-Dependent MoonTag Activation

Arabidopsis seedlings were germinated and grown at 24° C. degrees and then transferred for 24 hours at a temperature of 4° C., 18° C., 24° C., or 28° C. Results are shown in FIG. 12A and FIG. 13.

Hairy roots tomato lines transformed with MoonTag activating a luciferase reporter from a synthetic promoter were transferred to fresh media and allow to grow for seven days after which they were transferred for 24 hours. at a temperature of 4° C., 18° C., 24° C., or 28° C. Results are shown in FIG. 12B and FIG. 13.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A transcriptional activator system comprising:
a dCas protein;
a nanobody comprising an activator domain;
a binding polypeptide fused to the dCas protein, the binding polypeptide comprising an amino acid binding sequence designed to bind to the nanobody; and
a sgRNA.

2. The transcriptional activator system of claim 1, wherein the nanobody is llama GP41 (SEQ ID NO:2) and the amino acid binding sequence is GP41 (SEQ ID NO:1).

3. The transcriptional activator system of claim 1, wherein the nanobody further comprises a solubilizing domain.

4. The transcriptional activator of system of claim 3, wherein the solubilizing domain comprises GB1, sfGFP, or both.

5. The transcriptional activator system of claim 1, wherein the activator domain is VP64 (SEQ ID NO:4), TAL (SEQ ID NO:34), or a combination thereof.

6. The transcriptional activator system of claim 1, wherein the dCas protein is dCas9 SEQ ID NO:5).

7. The transcriptional activator of claim 1, wherein the binding polypeptide comprises at least five copies of the amino acid binding sequence.

8. A transcriptional activator system comprising:
a first nucleotide sequence that encodes a dCas protein;
a second nucleotide sequence that encodes a binding polypeptide comprising an amino acid binding sequence, the binding polypeptide fused to the dCas protein;
a third nucleotide sequence that encodes a nanobody, the nanobody designed to bind to the binding polypeptide;
a fourth nucleotide sequence that encodes an activator domain; and
a fifth nucleotide sequence that encodes a sgRNA sequence.

9. The transcriptional activator system of claim 8, wherein the nanobody is llama GP41 (SEQ ID NO:2) and the amino acid binding sequence is GP41 (SEQ ID NO:1).

10. The transcriptional activator system of claim 8, wherein the third nucleotide sequence further encodes a solubilizing domain, the solubilizing domain fused to the nanobody.

11. The transcriptional activator of system of claim 10, wherein the solubilizing domain comprises GB1, sfGFP, or both.

12. The transcriptional activator system of claim 8, wherein the activator domain is VP64 (SEQ ID NO:4), TAL (SEQ ID NO:34), or a combination thereof.

13. The transcriptional activator system of claim 8, wherein the dCas protein is dCas9 (SEQ ID NO:5).

14. The transcriptional activator of claim 8, wherein the binding polypeptide comprises at least five copies of the amino acid binding sequence.

15-21. (canceled)

22. A synthetic promoter influencing transcription of a target gene comprising:
a core promoter comprising:
a first region 15-20 nucleotides downstream from a transcription initiation site of the target gene; and
a second region 80-85 nucleotides upstream from the transcription initiation site of the target gene; and
a trans-activation region upstream from the transcription initiation site of the target gene, the trans-activation region comprising at least one sgRNA binding site.

23. The synthetic promoter of claim 22, wherein the core promoter comprises an Arabidopsis promoter.

24. The synthetic promoter of claim 23, wherein the core promoter comprises SEQ ID NO: 27, SEQ ID NO:28, SEQ ID NO:29, SEQ ID NO:30, SEQ ID NO:31, or SEQ ID NO:32.

25. The synthetic promoter of claim 22, wherein the trans-activation region comprises one or more additional sgRNA binding sites.

26. The synthetic promoter of claim 22, wherein the trans-activation region is SEQ ID NO. 24, SEQ ID NO:25, or SEQ ID NO:26.

27. A method comprising:
integrating the synthetic promoter of claim 22 into the genome of a cell;
providing the cell the transcriptional activator system of claim 1; and
allowing the cell to express the dCas protein, express the peptide, express the nanobody, transcribe the sgRNA, integrate the sgRNA with the dCas protein, pair the sgRNA with the sgRNA binding site, and initiate transcription of the target gene.

28. The method of claim 27, wherein the cell is a plant cell.

29. The method of claim 27, wherein the synthetic promoter comprises:
a core promoter selected from the group consisting of SEQ ID NO:24, ID NO: 25 and SEQ ID NO:26; and
a trans-activating region selected from the group consisting of SEQ ID NO:27, SEQ ID NO:28, SEQ ID NO:29, SEQ ID NO:30, SEQ ID NO:31 and SEQ ID NO:32.

30. The method of claim 27, further comprising integrating one or more syntenic promoters influencing the transcription of one or more additional target genes in the genome of the cell.

31. The method of claim 30, further comprising providing the cell with one or more sgRNA designed to pair with the one or more sgRNA binding sites on the one or more transcription activation region.
